(19) United States
(12) Patent Application Publication
(10) Pub. No.: US 2021/0002242 A1
(43) Pub. Date: Jan. 7, 2021

(54) DIACYLGLYCEROL LACTONE COMPOUND, PREPARATION METHOD THEREFOR, AND IMMUNOSTIMULATOR CONTAINING SAME AS ACTIVE INGREDIENT

(71) Applicant: ENZYCHEM LIFESCIENCES CORPORATION, Jecheon-si (KR)

(72) Inventors: Ki Young SOHN, Seoul (KR); Jae Wha KIM, Daejeon (KR); Sun Young YOON, Daejeon (KR); Chang Hyun YOO, Daejeon (KR); Jin Seon JEONG, Cheonan-si (KR)

(21) Appl. No.: 16/977,548

(22) PCT Filed: Mar. 8, 2019
(86) PCT No.: PCT/KR2019/002757

§ 371 Date: Sep. 2, 2020

(30) Foreign Application Priority Data
Mar. 12, 2018 (KR) 10-2018-0028871

(51) Int. Cl. C07D 307/32; A23L 33/00; A23L 29/00; A61P 31/04

(52) U.S. Cl. CPC C07D 307/32; A61P 31/04; A23L 29/035; A23L 33/30

(57) ABSTRACT

Disclosed are a novel diacylglycerol lactone compound for improving immunity and inhibiting infection by promoting neutrophil movement, a preparation method therefor, and an immunostimulator containing same as an active ingredient. The diacyloglycerol lactone compound is represented by chemical formula 1 of the specification. In chemical formula 1, R1 and R2 are respectively N and independently a C2-30 fatty acid group.

### TECHNICAL FIELD

The present invention relates to a diacylglycerol lactone compound, and more particularly, to a novel diacylglycerol lactone compound for promoting neutrophil migration to enhance immunity and to suppress infection, a method for preparing the same and an immunostimulator comprising the same as active ingredient.

### BACKGROUND ART

Immunity is a self-defense response that physiologically recognizes, eliminates and/or metabolizes exogenous and endogenous foreign substances in a body. The immune response can be classified into an innate immunity which is an initial immune response and an acquired immunity which is a late immune response. In the initial immune response, a host is protected by suppressing foreign substances (pathogens) by the activities of macrophages and natural killer cells (NK cells). At this time, the macrophages phagocytize foreign substances and produce and secrete TNF-α which is an active marker. NK cells destruct pathogen-infected cells by producing and secreting perforin which is an active marker. Subsequently, cytotoxic T lymphocytes, helper T lymphocytes, and B lymphocytes, which are involved in the acquired immunity, are activated to kill infected cells or produce antibodies thereby to protect the host. The cytotoxic T lymphocytes kill pathogen-infected cells by producing and secreting a lot of perforin like NK cells. B lymphocytes protect the host by producing antibodies, either dependent or independent of helper T lymphocytes. Inflammatory cytokines such as IL-6, IL-8, TNF-α and so on are substances that mediate the immune response and are known to be particularly involved in the initial immune response.

Generally, in case of immunodeficiency, resistance to infection is lowered, patients with inadequate antibody production cannot protect themselves against bacterial infection, and phagocytosis ability of neutrophils is also lowered. Further, in this case, since an activation of a complement system is also suppressed, leukocyte migration factors, etc. are not produced, so that the inflammation increases and viralemia occurs, causing the virus to spread to the central nervous system or elsewhere. In case of cancer patients, during chemotherapy or radiation treatment, not only cancer cells but also normal cells are affected, and the patient's immunity may rapidly decrease as a side effect.

Immunostimulators are drugs for treating congenital and acquired immunodeficiency, and immunoglobulin, interferon (INF), and the like are representatively used as the immunostimulators. Among these, a preparation obtained by concentrating and purifying IgG, which is one of human immunoglobulins, is used for the prevention and treatment of measles, chickenpox, hepatitis B, mumps, etc. However, the preparation has disadvantages of pain at the injection site and lowering blood pressure. Interferon was discovered as an antiviral factor, but afterwards, its cell proliferation inhibitory action, its immune modulating action, etc. were discovered, and it is used as an antiviral agent and an antitumor agent. However, the type p thereof has side effects such as fever, boredom, loss of appetite, local pain for injection, and alopecia, while the type a thereof may cause side effects such as reduction of white blood cells due to temporary suppression of bone marrow function.

### DISCLOSURE OF INVENTION

An object of the present invention is to provide a novel diacylglycerol lactone compound having an immunity enhancing effect and a method for preparing the same.

Another object of the present invention is to provide a diacylglycerol lactone compound for enhancing immunity and inhibiting infection by increasing the expression of IL-8 (CXCL8) and promoting neutrophil migration, and an immunostimulator containing the diacylglycerol lactone compound as an active ingredient.

In some embodiments for achieving the above objects, the present invention provides a diacylglycerol lactone compound represented by following formula 1.

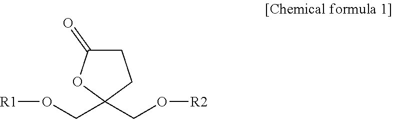

In Chemical formula 1, R1 and R2 each is independently a fatty acid residue of 2 to 30 carbon atoms.

In addition, the present invention provides an immunostimulator and a health functional food composition for immunity enhancement comprising the diacylglycerol lactone compound represented by Chemical formula 1 as an active ingredient. In addition, the present invention provides a method for enhancing immunity comprising administering an immunostimulator containing a diacylglycerol lactone compound represented by Chemical formula 1 as an active ingredient to a non-human subject.

The diacylglycerol lactone compound of the present invention is a novel compound having an immunity enhancing effect. It increases the expression of IL-8 (CXCL8) and promotes neutrophil migration, thereby enhancing immunity and inhibiting infection.

### BRIEF DESCRIPTION OF DRAWINGS

FIG. 1 is a graph showing an increase in the expression of CXCL8 (IL-8) in cells due to a diacylglycerol lactone compound according to the present invention.

FIG. 2 is a graph showing a bacterial infection inhibitory effect of the diacylglycerol lactone compound according to the present invention.

FIG. 3 is a photograph showing the results of the bacterial infection inhibition experiment of diacylglycerol lactone compound according to the present invention.

### BEST MODES FOR CARRYING OUT THE INVENTION

Hereinafter, with reference to the accompanying drawings, the present invention will be described in detail.

The present invention provides a novel diacylglycerol lactone compound represented by the following Chemical formula 1.

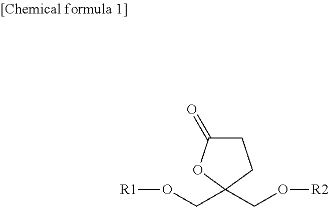

In Chemical formula 1, R1 and R2 each is independently a fatty acid residue of 2 to 30 carbon atoms, preferably 2 to 20 carbon atoms. Preferably R1 is a carbonyl group of 2 to 6 carbon atoms, preferably 2 to 3 carbon atoms (i.e., acetyl group or propionyl group), R2 is a fatty acid residue of 4 to 30 carbon atoms, preferably 4 to 20 carbon atoms. Specifically, R1 and R2 each is independently acetyl, propionyl, butyryl, isobutyryl, valeroyl, pivaloyl, 2-methylbutyryl, cyclopropanecarbonyl, cyclohexanecarbonyl, hexanoyl, heptanoyl, nonanoyl, dodecanoyl, myristoyl, palmitoyl, linoleoyl, oleoyl, linolenoyl, eicosanoyl, arachidonoyl and so on, preferably, acetyl, propionyl, butyryl, isobutyryl, cyclopropanecarbonyl, 2-methylbutyryl, pivaloyl, palmitoyl, linoleoyl and so on. The compounds of Chemical formula 1 is a racemic material or an optically active material.

Preferred examples of the diacylglycerol lactone compound represented by Chemical formula 1 may include a compound in which R1 or R2 is an acetyl group, and specific examples includes compounds represented by the following Chemical formulas 2a and 2b.

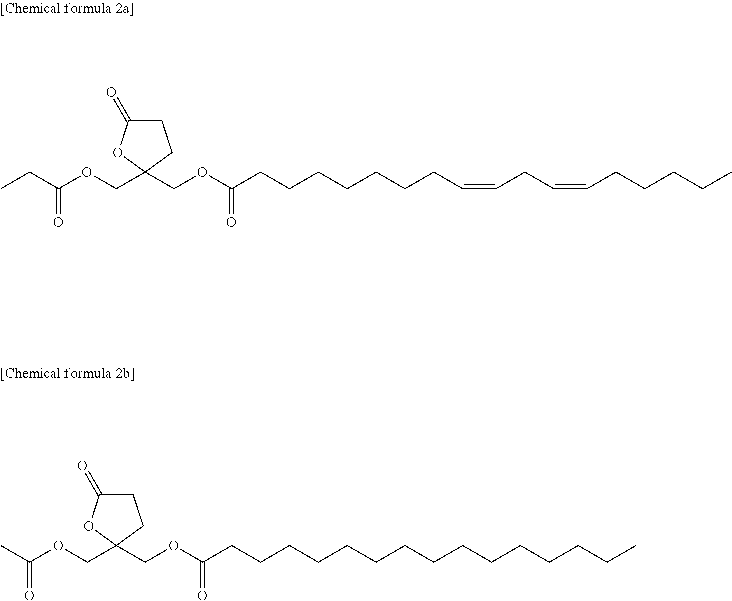

The diacylglycerol lactone compound represented by Chemical formula 1 can be prepared by the following Reactions 1 to 4.

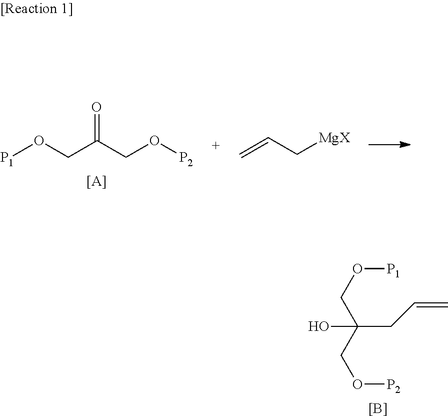

Reaction 1 is a reaction for preparing a compound represented by Chemical formula B by reacting the compound represented by Chemical formula A with CH₂═CH—CH₂—MgX (X is halogen atom), wherein P1 and P2 are protecting groups. The protecting groups may be selectively removed during a deprotection reaction, and each independently can be 4-metoxyphenyl, benzyl, tert-butyldiphenylsilane and so on. As the solvent for the reaction, an organic solvent may be used, and preferably tetrahydrofuran, diethyl ether, dioxane, etc., which are ethereal solvents, may be used. The equivalent of CH₂═CH—CH₂—MgX (X is a halogen atom, for example, Br or Cl, specifically CI) Grignard reagent is preferably 2 to 4 equivalents with respect to the reactant [A].

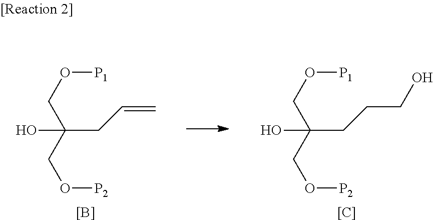

Reaction 2 is a reaction for producing a compound represented by Chemical formula C by carrying out hydroxylation reaction of the double bond of Chemical formula B compound. As the reaction solvent, tetrahydrofuran may be used 15 to 30 times, preferably 20 times by volume with respect to the weight of compound [B]. In the Reaction 2, boron reacts with the double bond (boration) and then hydroxylation reaction proceeds. The amount of borane dimethylsulfide (BMS) used for the formation of a ring ether is 1.5 to 2 equivalents, preferably 1.6 to 1.7 equivalents.

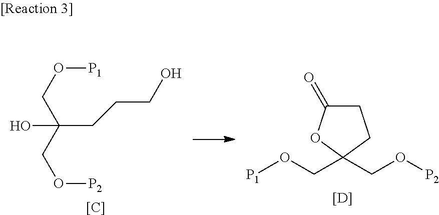

Reaction 3 is a reaction to obtain a compound represented by Chemical formula D by cyclizing a compound of Chemical formula C by lactone cyclization reaction. As the reaction solvent, methylene chloride may be used 3 to 10 times, preferably 5 times by volume with respect to the weight of compound [B]. The amount of pyridinium chlorochromate (C₅H₆NClCrO, PCC) used for lactone formation is 9 to 11 equivalents, preferably 10 equivalents of compound [B].

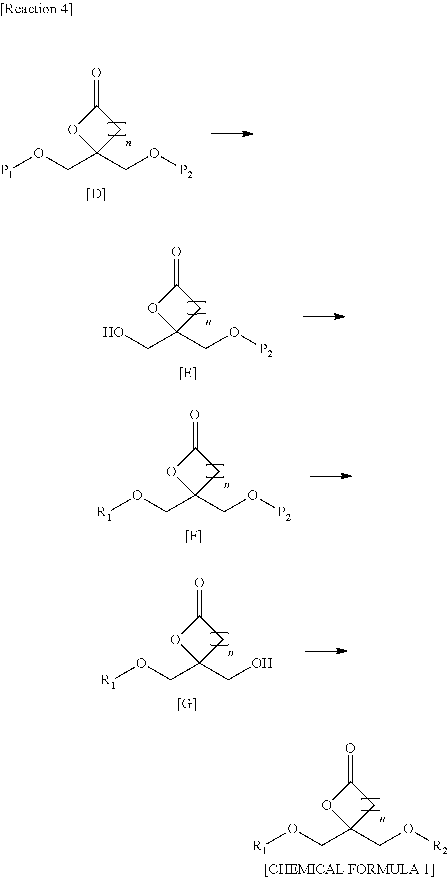

Reaction 4 is a reaction to obtain a compound represented by Chemical formula 1 by deprotecting and esterifying the compound of Chemical formula D (deprotection and esterification reaction). Selective deprotection reaction of one side chain and an esterification reaction are sequentially carried out. R1 and R2 are as defined in Chemical formula 1. Selective deprotection agents include Ceric Ammonium Nitrate (CAN), Boron trichloride (1M in MC, BCl₃), tetrabutyl ammoniumfluoride (TBAF) and so on, and the amount thereof is 2 to 4 equivalents, preferably 3 equivalents of compound [D], [F]. As a solvent, a mixed solvent composed of acetonitrile and purified water and a methylene chloride can be used, and the amount thereof is 15 to 30 times, preferably 24 times by volume with respect to the weight of Compound [D].

In the esterification reaction of Reaction 4, a fatty acid (R1OH or R2OH) having a carbon atom number of 2 to 30 can react with pivaloyl chloride in a non-polar organic solvent in the presence of an organic base, to prepare a mixed anhydride

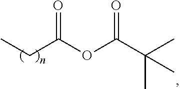

n=an integer of 0 to 20) of an activated form. Next, Compounds [E] and [G] and the mixed anhydride are reacted in the presence of 4-4-dimethylaminopyridine (4-dimethylamino pyridine, DMAP) to prepare the diacylglycerol lactone compound of Chemical formula 1.

The diacylglycerol lactone compound represented by Chemical formula 1 increases the expression of IL-8 (CXCL8) cytokine and promotes neutrophil migration from blood vessels to tissues, thereby enhancing immunity and inhibiting infection. Therefore, the diacylglycerol lactone compound of the present invention can be used to prevent or treat immune-related diseases. Examples of immune-related diseases that can be prevented or treated by the administration of the diacylglycerol lactone compound of the present invention may include various bacterial and viral infection diseases, acute or chronic inflammatory lung diseases, pneumonia, sepsis, and so on. As used herein, the term “prevention” or “preventing” includes any activity to suppress the decline of immunity or enhance immunity by administering the composition of the present invention. The term “treatment” or “treating” includes any activity to improve or beneficially alter the symptoms of immune-related diseases by the composition of the present invention.

The diacylglycerol lactone compound of the present invention increases the expression of CXCL8 (IL-8) in cells, promotes neutrophil migration, and inhibits bacterial infection bronchial fungal infection of animal models. Neutrophil, which is generally produced up to 101 per day in normal people, matures from the bone marrow, then circulates in blood vessels for about 8 hours, penetrates into the tissues, survives for several days, and dies or disappears. When a bacterial infection occurs, the immune cells that primarily remove bacteria are neutrophils. Vascular endothelial cells are activated by chemokines (CXCL8 or CXCL2, etc.) and various inflammatory factors secreted from damaged tissues in the area infected with bacteria, thereby circulating neutrophil cells in the blood vessels to move to the tissue and to removes bacteria present in infected tissue. In the experimental example of the present invention, it was confirmed that the diacylglycerol lactone compound of Chemical formula 1 increased the expression of CXCL8 chemokine (IL-8) in THP-1 cells, which are cells of the human macrophage family (Experimental Example 2-1), and the diacylglycerol lactone compound of Chemical formula 1 inhibited bacterial infection in a bacterial fungal lung infection mouse model (Experimental Example 4-1).

Diacylglycerol lactone compound of present invention may be used as an immunostimulator alone without mixing other substance, or in the form of a pharmaceutical composition containing the diacylglycerol lactone compound as an active ingredient. When diacylglycerol lactone compound of present invention is used in the pharmaceutical composition, conventional pharmaceutically acceptable carriers, excipients, or diluents can be included therein. The amount of diacylglycerol lactone compound in the pharmaceutical composition can be widely varied without specific limitation, and is specifically 0.0001 to 100.0 weight %, preferably, 0.001 to 95.0 weight %, more preferably 0.01 to 50 weight %, for example 1 to 20 weight %, with respect to the total amount of the composition.

The pharmaceutical composition may be formulated into any one selected from the group consisting of tablets, bolus, powders, granules, capsules, suspensions, liquid solutions, emulsions, syrups, sterilized aqueous solutions, non-aqueous solutions, suspensions, emulsions, freeze-dried agents, and suppositories and so on, and may be formulated into various forms for oral or non-oral administration. In formulating the composition, conventional excipients or diluents such as fillers, bulking agents, binders, wetting agents, disintegrating agents, and surfactants can be used. The solid formulation for oral administration includes tablet, bolus, powder, granule, capsule and so on, and such solid formulations can be prepared by mixing one or more of the components and at least one excipient such as starch, calcium carbonate, sucrose, lactose, gelatin, and so on. Besides the excipient, a lubricant such as Magnesium stearate and talc can also be used. The liquid formulation for oral administration includes suspension, liquid solutions, emulsion, syrup, and so on, and may include conventional diluents such as water and liquid paraffin or may include various excipients such as wetting agents, sweeting agents, flavoring agents, and preserving agents. The formulation for non-oral administration includes sterilized aqueous solution, non-aqueous solution, suspension, emulsion, freeze-dried formulation, suppository, and so on, and solvent for solution such as non-aqueous solution, suspension may include propylene glycol, polyethylene glycol, vegetable oils such as olive oil, and ester for syringe injection such as ethyl oleate. Base materials of the suppository may include witepsol, macrogol, tween 61, cacao butter, Laurin and glycerogelatin.

The composition of present invention can be administered in a pharmaceutically effective amount. The term “pharmaceutically effective amount” is used to refer to an amount that is sufficient to treat a disease at a reasonable benefit/risk ratio applicable to achieve a desired result in a medical treatment. The “pharmaceutically effective amount” can be determined according to the subject's category, age, sex, severity and type of disease, activity of drug, sensitivity to drug, administration time, administration route, excretion rate, duration of treatment, factors including concurrent drugs, and other factors well known in the medical field. The composition of the present invention can be administered alone or with other therapeutic agents sequentially or simultaneously. The composition of the present invention can be administered once or multiple times. It is important to administer an amount capable of obtaining the maximum effect in a minimum amount without side effects in consideration of all of the above factors, which can be easily determined by a person skilled in the art. The preferable amount of the composition of the present invention can be varied according to the condition and weight of patient, severity of disease, formulation type of drug, administration route and period of treatment. An appropriate total amount of administration per 1 day can be determined by a physician, and is generally about 0.001 to about 1000 mg/kg, preferably about 0.05 to 200 mg/kg, more preferable about 0.1 to about 100 mg/kg once a day or can be administered in divided doses multiple times daily. The compound or composition can be applied to any subject without specific limitation as long as it is an individual for the purpose of preventing immunity reduction, of enhancing immunity, or of treating an immune disease. For example, the composition of the present invention can be administered to not only human but also non-human animal (specifically mammals) such as monkey, dog, cat, rabbit, guinea pig, rat, mouse, cow, sheep, pig, goat, and birds and fishes, and so on. The composition of the present invention can be administered by conventional various methods, for example, by oral or rectum administration, or by intravenous (i.v.), intramuscular (i.m.), subcutaneous (s.c.), intrauterine dural or cerebrovascular injection.

In some embodiments, the present invention provides health functional food compositions for enhancing immunity, which comprises a diacylglycerol lactone compound of formula 1 as an active ingredient. Specifically, the diacylglycerol lactone compound of the present invention may be included in a health functional food composition for preventing immunity lowering, enhancing immunity, preventing or improving immune-related diseases. The term “improvement” or “improving” refers to any activity to improve or ameliorate the symptoms of an individual who is suspicious of an immune-related disease or developing an immune-related disease.

The health functional food composition may consist of only or substantially pure compound of the present invention or may include compound of the present invention together with other conventional ingredients of health functional food. The amount of the active ingredient in the health food composition can be determined suitably according to the intended use. Generally, when the compound of the present invention is included in food or beverages, the amount of the composition according to the present invention is preferably less than 15 weight %, more preferably less than 10 weight %, with respect to the total amount of the raw material. In case of a long term use for the purpose of the health control and hygiene, the amount can be less than the above range. Since there is no problem in terms of safety, amount of the active component is greater than the above range.

Foods to which the compound of the present invention can be added are not limited, and include various foods, for example, meats, sausages, breads, chocolates, candies, snacks, pizzas, noodles, gums, daily products such as ice creams, soups, beverages, teas, drinks, alcoholic beverages, vitamin complexes and any health functional food, and also include food used as feed for animals. When the health functional food composition of present invention is used in the beverage product, the beverage product may include sweeting agents, flavoring agents or natural carbohydrates. Examples of natural carbohydrates include monosaccharides such as glucose and fructose, disaccharides such as maltose and sucrose, polysaccharides such as dextrin and cyclodextrin, and sugar alcohols such as xylitol, sorbitol and erythritol. The amount of carbohydrate in the beverage composition can be widely varied without specific limitation, and is preferably 0.01 to 0.04 g, more preferably, 0.02 to 0.03 g per 100 ml of the beverage. Examples of sweeting agents include natural sweeteners such as thaumatin and stevia extract and artificial sweeteners such as saccharin and aspartame. In addition to the above, the health functional food composition of the present invention may include various nutrients, vitamins, electrolytes, flavoring agents, colorants, pectic acid and salts thereof, alginic acid and salts thereof, organic acids, protective colloidal thickening agents, pH controlling agents, stabilizing agents, preserving agents, glycerin, alcohol, carbonizing agents used in carbonated beverages and so on. Moreover, the health functional food composition of the present invention may include fruits, as used in preparing natural fruit juices and fruit juice beverages and vegetable beverages.

In some embodiments, the present disclosure provides methods for enhancing immunity or preventing or treating an immune-related disease, comprising administering the pharmaceutical composition to a patient in need thereof. The term “a patient in need” includes any animal including human that suffers from immune-related disease or can develop immune-related disease. Immune-related disease can be treated or prevented by administering an effective amount of a pharmaceutical composition containing a compound of the present invention or containing the compound of the present invention and pharmaceutically acceptable salt thereof to a patient in need thereof. The term “administration” means introducing the pharmaceutical composition of the present invention to a patient in need by any suitable method. The composition of the present disclosure can be administered by conventional various methods, for example, by oral or non-oral administration as far as the target organization can be reached. In some embodiments, the method of the present disclosure comprises administering a therapeutically effective amount of a pharmaceutical composition comprising diacylglycerol lactone compound of formula I to a patient in need thereof. An appropriate total amount of administration per 1 day can be determined by a physician, and is generally about 0.001 to about 1000 mg/kg, preferably, about 0.05 to 200 mg/kg, more preferably about 0.1 to about 100 mg/kg. The total administration amount per day can be administered once a day or can be administered in divided doses multiple times daily. However, the specific therapeutically effective amount of pharmaceutical composition administered to a particular patient can be varied depending on the type and degree of the response to be achieved in the treatment, the specific composition, including whether another agent is included in the composition, the patient's age, body weight, general health status, sex, diet, administration time, administration route, the ratio of composition, treatment period, other drugs used together in the treatment and a variety of factors well known in the medical field.

### Embodiments for Carrying Out the Invention

Hereinafter, the present invention is described in more detail through examples. The following example is only to help the understanding of the present invention, and the present invention is not limited by the following examples.

### [Example 1] Synthesis of Diacylglycerol Lactone Compound (EC-A129)

A. As shown in Reaction 1a below, 50 g (402.77 mmole) of 4-methoxyphenol was dissolved in 1500 ml of acetone. Then, 278 g (2013.8 mmole) of K₂CO₃ was added and stirred at room temperature for 30 minutes. 126 ml (1611.1 mmole) of epichlorohydrin was added to the 4-methoxyphenol solution, the temperature was raised to 60 to 65° C., and refluxed for 72 hours. The reaction was confirmed by TLC (ethylacetate(EA):hexane(Hex)=1:9). When the reaction was completed, the reaction solution was filtered by a celite filter, the filtrate was concentrated. The concentrate was purified with a flash column (eluent:ethyl acetate(EA):hexane(Hex)=1:10 (volume ratio) mixture) to obtain 68 g of the target compound (yield: 97.3%)

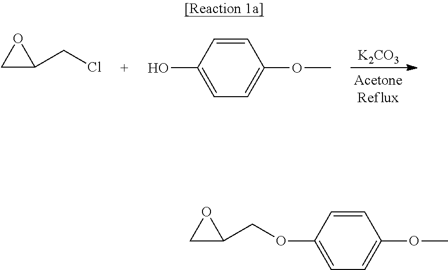

B. As shown in Reaction 1b below, 0.73 ml (7.0755 mmole) of benzylalcohol was dissolved in 4 ml of dimethylformamide (DMF). 283 mg (7.0755 mmole) of 60%-NaH was slowly added and stirred at room temperature for 30 minutes. 850 mg (4.717 mmol) of the product of Reaction 1a (SM) was dissolved in 3 ml of dimethylformamide (DMF). The dissolved solution was slowly added dropwise to the reaction solution, followed by stirring at 80° C. for 3 hours. The reaction was confirmed by TLC (EA:Hex=1:2). When the reaction was completed, H₂O was added to the reaction solution to quench the reaction, extracted with ethyl acetate (EA)/H₂O. Thereafter the organic layer was washed with purified water 3 times, water was removed with MgSO₄, and then concentrated. The concentrate was purified with a flash column (eluent:ethyl acetate (EA):hexane (Hex)=1:4 (volume ratio) mixture) to obtain 1.07 g of the target compound (yield: 78.3%).

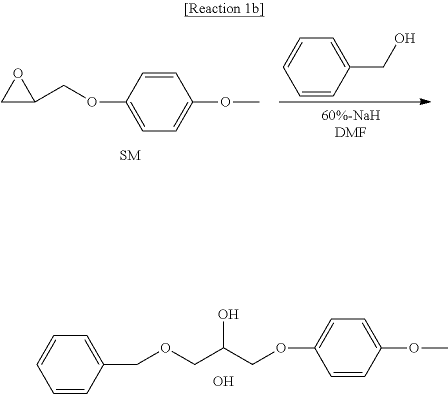

C. As shown in Reaction 1c below, 52.77 g (297.79 mmole) of pyridinium chlorochromate (PCC) and 52.77 g of celite were added in 233.5 ml of methylene chloride (MC) and stirred. 23.55 g (81.67 mmole) of the product (SM) of Reaction 1b was dissolved in 81.2 ml of methylene chloride (MC) and it was slowly added dropwise. It was stirred at room temperature for 24 hours. The reaction was confirmed by TLC (EA:Hex=1:2). When the reaction was completed, it was filtered through a celite filter, and the filtrate was concentrated. The concentrate was purified with a flash column (eluent:ethyl acetate(EA):methylene chloride (MC):hexane(Hex)=1:1:4 (volume ratio) mixture) to obtain 14.2 g of the target compound (yield: 60.7%).

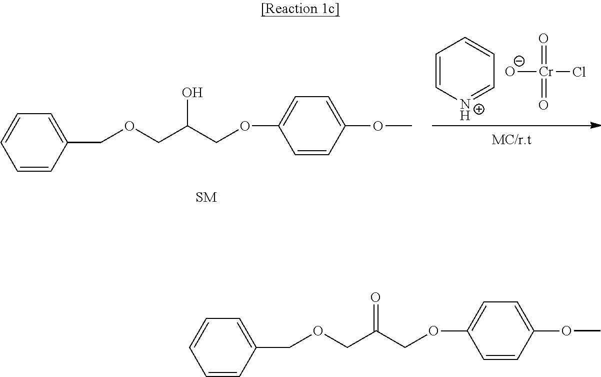

D. As shown in Reaction 1d below, 370 mg (1.29 mmole) of the product (SM) of Reaction 1c was dissolved in 1.3 ml of tetrahydrofuran (THF). Then, after bubbling with nitrogen gas (N2), it was cooled to 0° C. 1.94 ml (3.877 mmle) of allylmagnesium chloride (2M in THF) was slowly added dropwise and stirred at room temperature for 2 hours. The reaction was confirmed by TLC (EA:Hex=1:4). When the reaction was completed, a dilute aqueous hydrochloric acid solution was added to the reaction solution to quench the reaction. Then, it was extracted with ethyl acetate(EA)/H2O and then concentrated after removing moisture with magnesium sulfate (MgSO₄). The concentrate was purified with a flash column (eluent:ethyl acetate(EA):hexane(Hex)=1:7 (volume ratio) mixture) to obtain 250 mg of the target compound (yield: 59%).

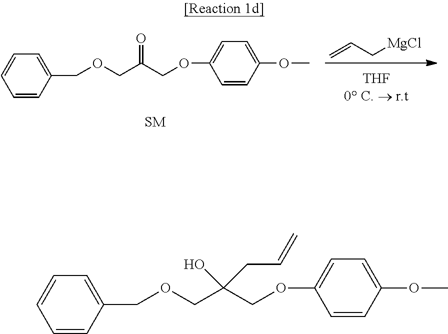

E. As shown in Reaction 1e below, 6.5 g (19.793 mmol) of the product (SM) of Reaction 1d was dissolved in 130 ml of tetrahydrofuran (THF). Then, after bubbling with nitrogen gas (N2), it was cooled to −78° C. 16 ml (31.977 mmole) of borane dimethyl sulfide solution (2M in THF, BH₃Me₂S) was slowly added dropwise and stirred at the same temperature for overnight. The reaction was confirmed by TLC (EA:Hex=1:2). When the reaction was completed, methanol (MeOH) was added to the reaction solution to quench the reaction, followed by concentration. 42.6 g (197.93 mmole) of pyridine chlorochromate (PCC) and 42.6 g of celite were added in 32.5 ml of methylene chloride (MC) and stirred. A solution in which the concentrated reactant SM2 was dissolved in an appropriate amount of methylene chloride (MC) was added and stirred at room temperature for 5 hours. The reaction was confirmed by TLC (EA:Hex=1:2). When the reaction was completed, it was filtered through a celite filter, and the filtrate was concentrated. The concentrate was purified with a flash column (eluent:ethyl acetate(EA):hexane(Hex)=1:4 (volume ratio) mixture) to obtain 4.56 g of the target compound (yield: 67.3%).

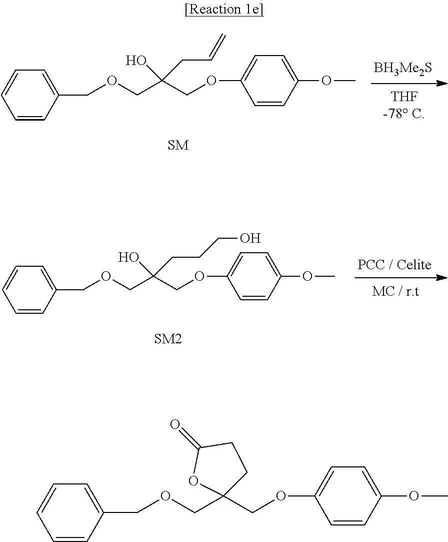 [SMILES: C=CCC(O)(COCc1ccccc1)COc1ccc(OC)cc1 ; COc1ccc(OCC(O)(CCCO)COCc2ccccc2)cc1 ; COc1ccc(OCC2(COCc3ccccc3)CCC(=O)O2)cc1]

F. As shown in Reaction 1f below, 2.94 g (8.59 mmol) of the product (SM) of Reaction 1e was dissolved in 70 ml of acetonitrile mixed solution (acetonitrile (ACN)/H₂O=8:2 (volume ratio) mixture). Then, it was cooled to 0° C. 14.2 g (25.76 mmole) of ceric ammonium nitrate (CAN) was added at the same temperature. The reaction was confirmed by TLC (EA:Hex=1:2). When the reaction was completed, a saturated sodium hydrogen carbonate solution (Saturated NaHCO₃aq. Soln.) was added to the reaction solution to quench the reaction. The reaction mixture was heated to room temperature and extracted with ethyl acetate(EA)/H₂O. After removing moisture with magnesium sulfate (MgSO₄), it was concentrated. It was obtained 2.8 g of the target compound (yield: 137.9%).

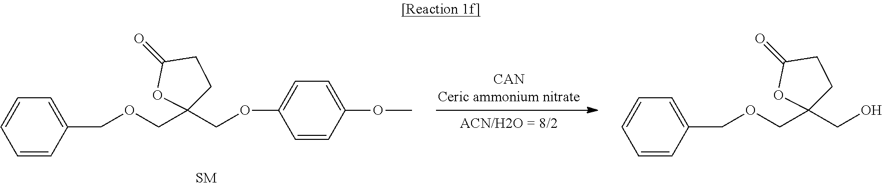 [SMILES: COc1ccc(OCC2(COCc3ccccc3)CCC(=O)O2)cc1 ; O=C1CCC(CO)(COCc2ccccc2)O1]

G. As shown in Reaction 1 g below, 5.88 g (22.945 mmole) of palmitic acid and 2.88 ml (22.945 mmole) of pivaloyl chloride were added in 58.8 ml of Methylene chloride (MC) and it was cooled to 0° C. While maintaining the same temperature, 7.4 ml (52.95 mmol) of triethylamine (TEA) was slowly added dropwise, followed by stirring at the same temperature for 30 minutes. 1.47 g (17.65 mmole) of the product (SM) of Reaction 1f was added, and 216 mg (1.765 mmole) of 4-dimethylaminopyridine (DMAP) was added, and then the temperature was raised to room temperature. Then, it was stirred at the same temperature for 2 hours. The reaction was confirmed by TLC (EA:Hex=1:2). When the reaction was completed, the reaction solution was extracted with an aqueous potassium hydroxide solution (aq. KOH soln.)/methylene chloride (MC), and extracted with a hydrochloric acid solution (HCl soln.)/methylene chloride (MC), and then concentrated. The concentrate was purified with a flash column (eluent:ethyl acetate (EA):hexane(Hex)=1:4:5 (volume ratio) mixture) to obtain 1.9 g of the target compound (yield: 22.6%).

Alternatively, SM (2.8 g, 11.85 mmole, 1 eq.)/Palmitic acid (1.3 eq.)/N,N′-dicyclohexylcarbodiimide (DCC, 1.3 eq.)/DMAP (0.1 eq)/MC (10 times of Palmitic acid) was reacted. The reaction was confirmed by TLC (EA:Hex=1:2). When the reaction was complete, the solvent was concentrated. Then, it was filtered after slurrying with an appropriate amount of hexane, and concentrated. The concentrate was purified with a flash column (eluent:ethyl acetate(EA):hexane(Hex)=1:7 (volume ratio) mixture) to obtain 3.66 g of the target compound (yield: 65%).

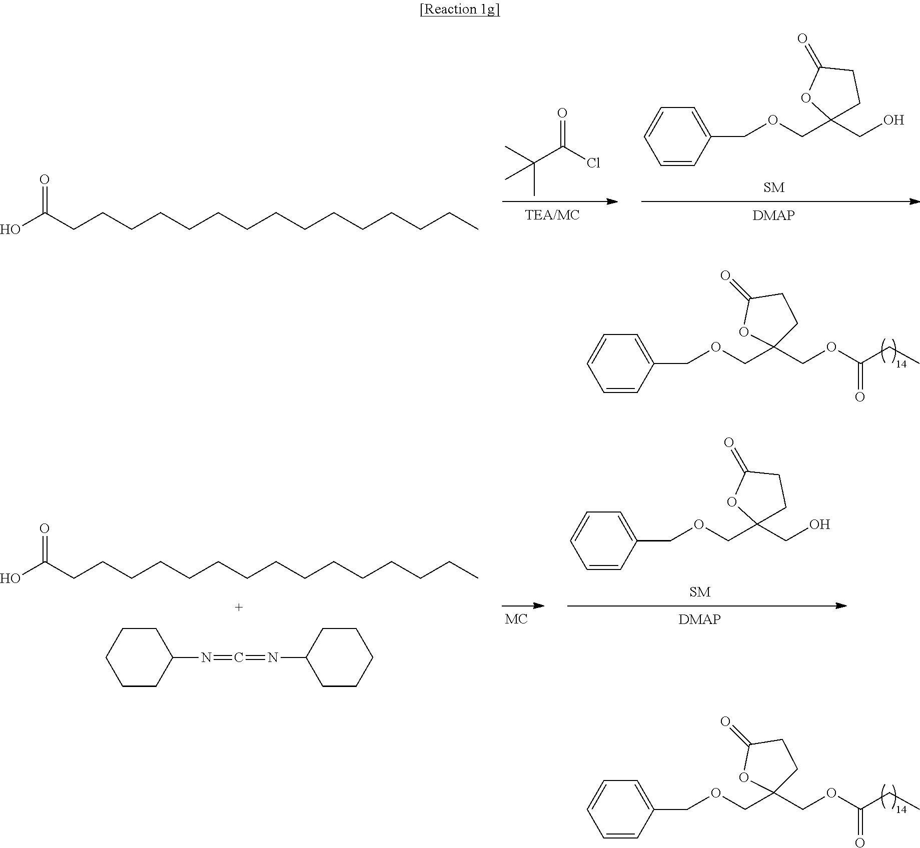

H. As shown in Reaction 1h below, 3.66 g (7.71 mmol) of the product (SM) of Reaction 1 g was added in 38 ml of methylene chloride (MC). Then, after bubbling with nitrogen gas (N₂), it was cooled to −78° C. While maintaining the same temperature, 23 ml (23.11 mmol) of boron trichloride (1M in MC, BCl₃) was slowly added dropwise, followed by stirring at the same temperature for 2 hours. The reaction was confirmed by TLC (EA:Hex=1:1). When the reaction was completed, a saturated sodium hydrogen carbonate solution (Saturated NaHCO₃aq. Soln.) was added to the reaction solution to quench the reaction. After extraction with methylene chloride (MC)/H₂O, it was concentrated. The concentrate was purified with a flash column (eluent:ethyl acetate(EA):hexane(Hex)=1:2 (volume ratio) mixture) to obtain 2.68 g of the target compound (yield: 90%).

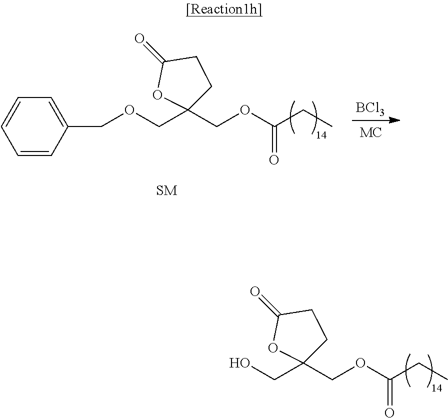

I. As shown in Reaction 1i below, 100 g (0.26 mmole) of the product (SM) of Reaction 1h, 72.5 mL (0.52 mmole) of triethylamine (TEA) and 3.17 mg (0.026 mmole) of 4-dimethylaminopyridine (DMAP) were added in 10 ml of methylene chloride (MC) and stirred at room temperature for 30 minutes. 24 mL (0.338 mmole) of acetyl chloride was slowly added dropwise, and it was stirred at the same temperature for 2 hours. The reaction was confirmed by TLC (EA:Hex=1:2). When the reaction was completed, the reaction solution was extracted with an aqueous potassium hydroxide solution (aq. KOH soln.)/methylene chloride (MC), and extracted with a hydrochloric acid solution (HCl soln.)/methylene chloride (MC), and then concentrated. The concentrate was purified with a flash column (eluent:ethyl acetate(EA):hexane(Hex)=1:3 (volume ratio) mixture) to obtain 87.4 mg of the target compound (yield: 78.8%).

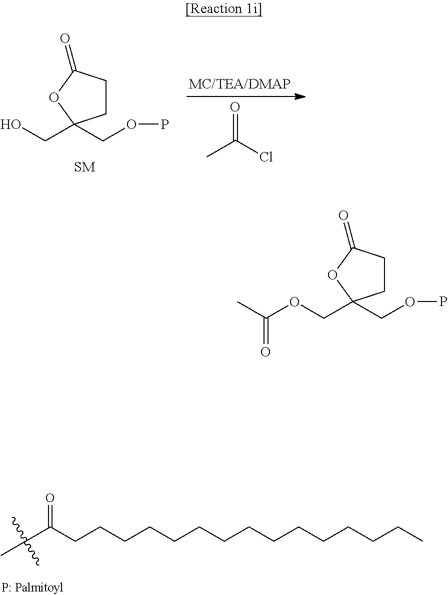

### [Example 2] Synthesis of Diacylglycerol Lactone Compound (EC-A51)

A. As shown in Reaction 2a below, 350 ml of pyridine, 10 g (111.01 mmole) of dihydroxyacetone and 3.53 g (28.86 mmole) of 4-dimethylaminopyridine (DMAP) were dissolved. Then, after bubbling with nitrogen gas (N2), it was cooled to 0° C. While maintaining the same temperature, 30.51 g (111.01 mmole) of tert-butyl(chloro)diphenylsilane (TBDPSCI) was slowly added dropwise. The reaction was stirred at the same temperature for 15 minutes, and heated to 25 to 30° C. It was stirred for 16 hours. The reaction was confirmed by TLC (PE:EA=10:1). When the reaction was completed, extraction was carried out 4 times with ethyl acetate(EA)/purified water. The organic layer is sequentially washed with 1M-hydrochloric acid and brine soln., and the organic layer is dehydrated with sodium sulfate (Na₂SO₄) and concentrated. The concentrate was purified with a flash column (eluent:Petroleum ether (PE):ethyl acetate(EA)=50:1 (volume ratio) mixture) to obtain 7 g of the target compound (yield: 19.2%).

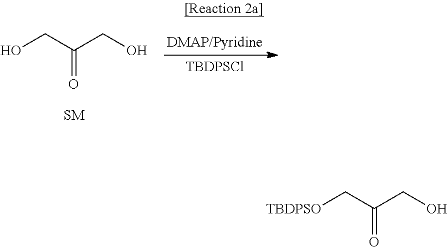

B. As shown in Reaction 2b below, 361.21 mg (4.57 mmole) of pyridine, 300 mg (0.9133 mmole) of the product (SM) of Reaction 2a and 16.74 mg (0.13699 mmole) of 4-dimethylaminopyridine (DMAP) were dissolved in 10 ml of methylene chloride (MC). Then, after bubbling with nitrogen gas (N2), it was cooled to 0° C. While maintaining the same temperature, 78.86 mg (1 mmole) of acetyl chloride was slowly added dropwise. The reaction was stirred at the same temperature for 5 minutes, and heated to 25 to 30° C. It was stirred for 4 hours. The reaction was confirmed by TLC (PE:EA=5:1). When the reaction was completed, ice water was added to the reaction and stirred for 5 minutes. It was extracted 4 times with ethyl acetate(EA)/purified water. The organic layer is sequentially washed with 1M-hydrochloric acid and brine soln., and the organic layer is dehydrated with sodium sulfate (Na₂SO₄) and concentrated. The concentrate was purified with a flash column (eluent:Petroleum ether (PE):ethyl acetate(EA)=50:1 (volume ratio) mixture) to obtain 200 mg of the target compound (yield: 59.1%).

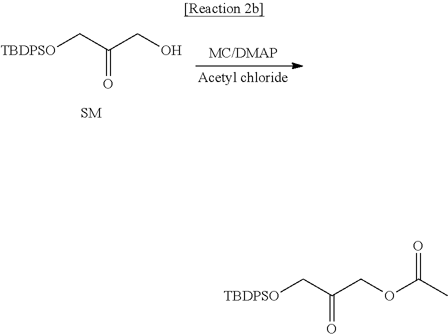

C. As shown in Reaction 2c below, 250 mg (0.6748 mmole) of the product (SM) of Reaction 2b was dissolved in 2 ml of tetrahydrofuran (THF). Then, after bubbling with nitrogen gas (N2), it was cooled to −70° C. While maintaining the same temperature, 337.3 mL (0.6748 mmole) of allylmagnesium chloride (2M in THF) was slowly added dropwise. The reaction was stirred at the same temperature for 15 minutes and heated to 25 to 30° C. It was stirred for 3 hours. The reaction was confirmed by TLC (PE:EA=10:1). When the reaction was completed, ice water was added to the reaction and stirred for 5 minutes. It was extracted 4 times with ethyl acetate(EA)/purified water. The organic layer is sequentially washed with 1M-hydrochloric acid and brine soln., and the organic layer is dehydrated with sodium sulfate (Na₂SO₄) and concentrated. The concentrate was purified with a flash column (eluent:Petroleum ether(PE):ethyl acetate(EA)=50:1 (volume ratio) mixture) to obtain 100 mg of the target compound (yield: 28.74%).

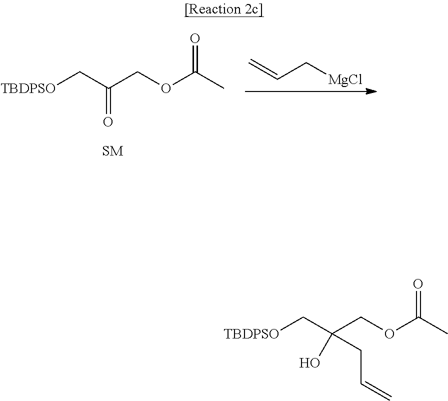

D. As shown in Reaction 2d below, 2 g (4.85 mmole) of the product (SM) of Reaction 2c was dissolved in 50 ml of tetrahydrofuran (THF). Then, after bubbling with nitrogen gas (N₂), it was cooled to −78° C. While maintaining the same temperature, 970 mL (9.7 mmole) of boranedimethylsulfide (10M in THF) was slowly added dropwise. The reaction was stirred at the same temperature for 30 minutes and heated to 25 to 35° C. It was stirred for 15.5 hours. The reaction was confirmed by TLC (PE:EA=10:1). When the reaction was completed, 80 ml of methanol was added to quench the reaction. The organic layer was concentrated to obtain 2.5 g of the target compound. This reaction product was used directly in the next reaction.

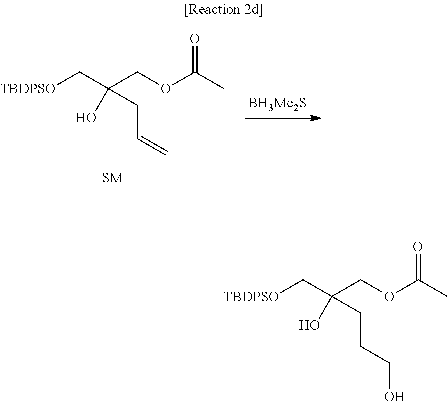

E. As shown in Reaction 2e below, 110 mg (0.25545 mmole) of the product (SM) of Reaction 2d was dissolved in 10 ml of methylene chloride (MC). Then, after bubbling with nitrogen gas (N2), it was heated to 25 to 30° C. While maintaining the same temperature, 550.65 mg (2.55 mmole) of pyridinium chlorochromate (PCC) was added to one portion. The reaction solution was stirred at the same temperature for 36 hours. The reaction was confirmed by TLC (PE:EA=2:1). When the reaction was completed, extraction was carried out 4 times with methylene chloride (MC)/purified water. The organic layer was washed with and brine soln., and the organic layer is dehydrated with sodium sulfate (Na₂SO₄) and concentrated. The concentrate was purified with a flash column (eluent:Petroleum ether(PE):ethyl acetate(EA)=5:1 (volume ratio) mixture) to obtain 60 mg of the target compound (yield: 44.05%).

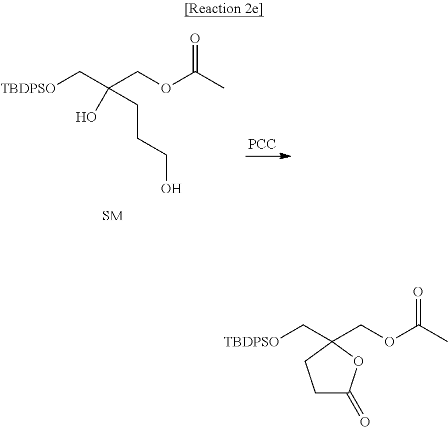

F. As shown in Reaction 2f below, 200 mg (0.46885 mmole) of the product (SM) of Reaction 2e was dissolved in 2 ml of tetrahydrofuran (THF). Then, after bubbling with nitrogen gas (N2), it was heated to 25 to 30° C. While maintaining the same temperature, 609.51 mL (1.3 eq.) of tetrabutyl ammoniumfluoride (1M in THF, TBAF) was added to one portion. The reaction solution was stirred at the same temperature for 2 hours. The reaction was confirmed by TLC (PE:EA=10:1). When the reaction was completed, the reaction solution was filtered and concentrated. The concentrate was purified with a flash column (eluent:Petroleum ether(PE):ethyl acetate(EA)=10:1 (volume ratio) mixture) to obtain 60 mg of the target compound (yield: 54.4%).

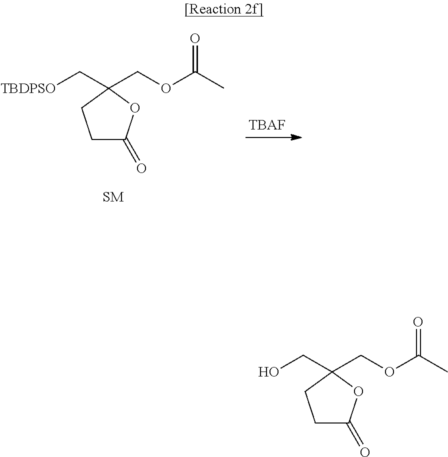

G. As shown in Reaction 2 g below, 19.96 mg (0.10608 mmole) of the product (SM) of Reaction 2f and 35 mg (0.1248 mmole) of linoleic acid were dissolved in 2 ml of methylene chloride (MC). Then, after bubbling with nitrogen gas (N2), it was heated to 25 to 35° C. While maintaining the same temperature, 30.9 mg (0.14976 mmole) of N,N′-dicyclohexylcarbodiimide (DCC) and 3.05 mg (0.02496 mmole) of 4-dimethylaminopyridine (DMAP) were added to one portion. The reaction was stirred at the same temperature for 16 hours. The reaction was confirmed by TLC (PE:EA=10:1). When the reaction was completed, ice water was added to the reaction solution and stirred for 5 minutes. It was extracted 4 times with ethyl acetate(EA)/purified water. The organic layer was washed with brine soln. The organic layer is dehydrated with sodium sulfate (Na₂SO₄) and concentrated. The concentrate was purified with a flash column (eluent:Petroleum ether(PE):ethyl acetate(EA)=10:1 (volume ratio) mixture) to obtain 20 mg of the target compound (yield: 33.79%).

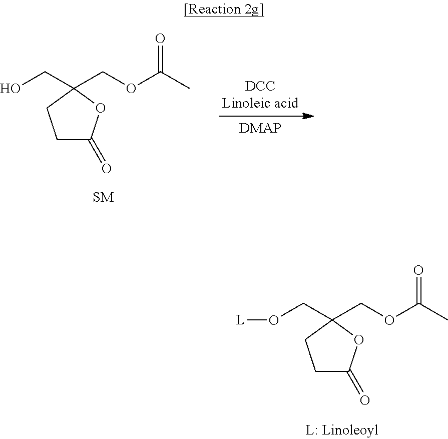

### [Example 3] Synthesis of Diacylglycerol Lactone Compound (EC-A52)

A. As shown in Reaction 3a below, 350 ml of pyridine, 10 g (111.01 mmole) of dihydroxyacetone and 3.53 g (28.86 mmole) of 4-dimethylaminopyridine (DMAP) were dissolved. Then, after bubbling with nitrogen gas (N₂), it was cooled to 0° C. While maintaining the same temperature, 30.51 g (111.01 mmole) of tert-butyl(chloro)diphenylsilane (TBDPSCI) was slowly added dropwise. The reaction solution was stirred at the same temperature for 15 minutes and heated to 25 to 30° C. It was stirred for 16 hours. The reaction was confirmed by TLC (PE:EA=10:1). When the reaction was completed, extraction was carried out 4 times with ethyl acetate(EA)/purified water. The organic layer is sequentially washed with 1M-hydrochloric acid and brine soln., and the organic layer is dehydrated with sodium sulfate (Na₂SO₄) and concentrated. The concentrate was purified with a flash column (eluent:Petroleum ether(PE):ethyl acetate(EA)=50:1 (volume ratio) mixture) to obtain 7 g of the target compound (yield: 19.2%).

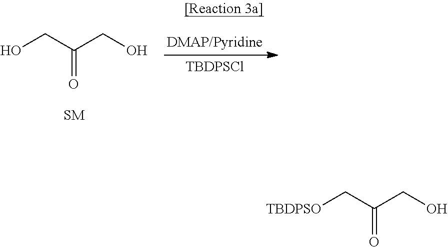

B. As shown in Reaction 3b below, 6 g (76.1 mmole) of pyridine, 5 g (15.22 mmole) of the product (SM) of Reaction 3a and 185.94 mg (1.52 mmole) of 4-dimethylaminopyridine (DMAP) were dissolved in 150 ml of methylene chloride (MC). Then, after bubbling with nitrogen gas (N₂), it was heated to 25 to 35° C. While maintaining the same temperature, 4.18 g (15.22 mmole) of palmitoyl chloride was slowly added dropwise. The reaction solution was stirred at the same temperature for 5 minutes, and heated to 25 to 35° C. It was stirred for 16 hours. The reaction was confirmed by TLC (PE:EA=10:1). When the reaction was completed, ammonium chloride aqueous solution (NH₄Cl soln.) was added to the reaction solution to quench the reaction. Then, it was extracted 3 times with ethyl acetate(EA)/purified water. The organic layer was washed with brine soln. The organic layer was dehydrated with sodium sulfate (Na₂SO₄) and concentrated. The concentrate was purified with a flash column (eluent:Petroleum ether(PE):ethyl acetate(EA)=50:1 (volume ratio) mixture) to obtain 4 g of the target compound (yield: 41.72%).

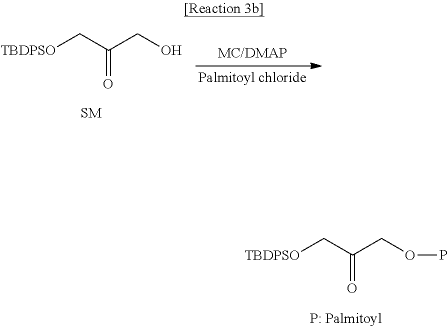

C. As shown in Reaction 3c below, 3.15 g (5.55 mmole) of the product (SM) of Reaction 3b was dissolved in 50 ml of tetrahydrofuran (THF). Then, after bubbling with nitrogen gas (N₂), it was cooled to 0° C. While maintaining the same temperature, 1.61 g (11.1 mmole) of allylmagnesium bromide was slowly added dropwise. The reaction solution was stirred at the same temperature for 15 minutes and heated to 25 to 30° C. It was stirred for 16 hours. The reaction was confirmed by TLC (PE:EA=10:1). When the reaction was completed, it was extracted 3 times with ethyl acetate(EA)/purified water. The organic layer was washed with brine soln. The organic layer was dehydrated with sodium sulfate (Na₂SO₄) and concentrated. The concentrate was purified with a flash column (eluent:Petroleum ether(PE):ethyl acetate(EA)=20:1 (volume ratio) mixture) to obtain 1.5 g of the target compound (yield: 44.32%).

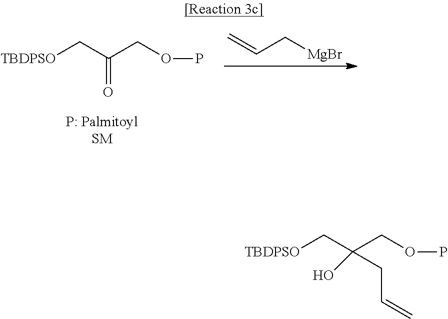

D. As shown in Reaction 3d below, 1.2 g (2.91 mmole) of the product (SM) of Reaction 3c was dissolved in 30 ml of tetrahydrofuran (THF). Then, after bubbling with nitrogen gas (N₂), it was cooled to −78° C. While maintaining the same temperature, 662.87 mg (8.73 mmole) of boranedimethylsulfide was slowly added dropwise. The reaction solution was stirred at the same temperature for 1 hour and heated to 25 to 35° C. It was stirred for 15 hours. The reaction was confirmed by TLC (PE:EA=10:1). When the reaction was completed, 80 ml of methanol was added to the reaction solution to quench the reaction. The organic layer was concentrated to obtain 1.3 g of the target compound. This reaction product was used directly in the next reaction.

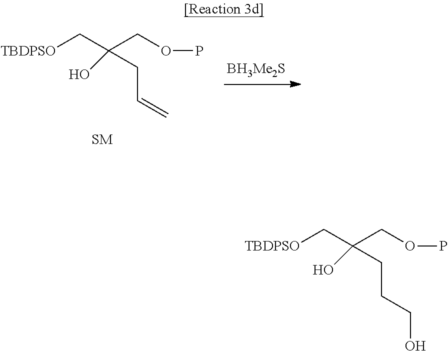

E. As shown in Reaction 3e below, 150 mg (0.23924 mmole) of the product (SM) of Reaction 3d was dissolved in 15 ml of methylene chloride (MC). Then, after bubbling with nitrogen gas (N₂), it was heated to 25 to 30° C. While maintaining the same temperature, 361 mg (1.67 mmole) of pyridinium chlorochromate (PCC) was added to one portion. The reaction solution was stirred at the same temperature for 48 hours. The reaction was confirmed by TLC (PE:EA=5:1). When the reaction was completed, the reaction product was concentrated to obtain 150 mg of the target compound.

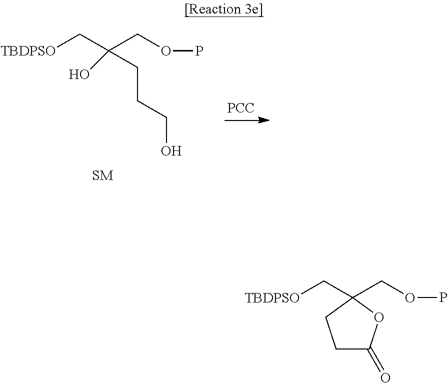

F. As shown in Reaction 3f below, 1.5 g (2.41 mmole) of the product (SM) of Reaction 3e was dissolved in 5 ml of tetrahydrofuran (THF). Then, after bubbling with nitrogen gas (N₂), it was heated to 25 to 35° C. While maintaining the same temperature, 3.13 mL (1.3 eq.) of tetrabutyl ammoniumfluoride (1M in THF, TBAF) was added to one portion. The reaction solution was stirred at the same temperature for 2 hours. The reaction was confirmed by TLC (PE:EA=5:1). When the reaction was completed, it was extracted 3 times with ethyl acetate(EA)/purified water. The organic layer was washed with brine soln., and the organic layer was dehydrated with sodium sulfate (Na₂SO₄) and concentrated. The concentrate was purified with a flash column (eluent:Petroleum ether (PE):ethyl acetate(EA)=5:1 (volume ratio) mixture) to obtain 250 mg of the target compound (yield: 24.28%).

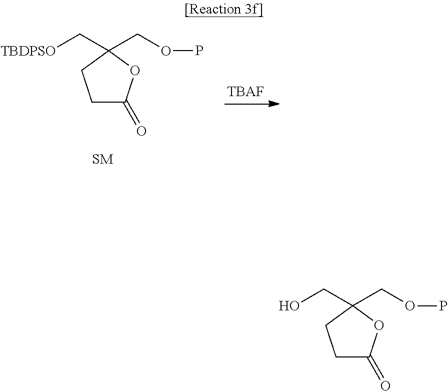

G. As shown in Reaction 3 g below, 250 mg (0.65011 mmole) of the product (SM) of Reaction 3f and 182.32 mg (0.65011 mmole) of linoleic acid were dissolved in 5 ml of methylene chloride (MC). Then, after bubbling with nitrogen gas (N₂), it was heated to 25 to 35° C. While maintaining the same temperature, 160.96 mg (0.78013 mmole) of N,N′-Dicyclohexylcarbodiimide (DCC) and 7.94 mg (0.06501 mmole) of 4-dimethylaminopyridine (DMAP) were added to one portion. The reaction solution was stirred at the same temperature for 16 hours. The reaction was confirmed by TLC (PE:EA=5:1). When the reaction was completed, the reaction solution was filtered and concentrated. The concentrate was purified with a flash column (eluent:Petroleum ether(PE):ethyl acetate(EA)=5:1 (volume ratio) mixture) to obtain 100 mg of the target compound (yield: 22.59%).

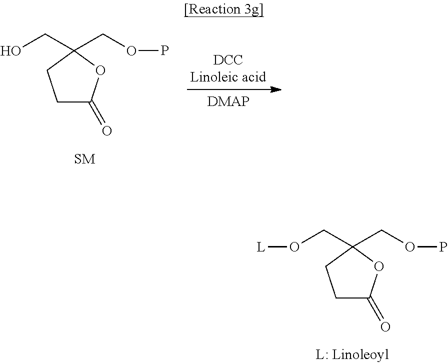

### [Example 4] Synthesis of Diacylglycerol Lactone Compound (EC-A115)

A. As shown in Reaction 4a below, 65 mg (0.275 mmole) of the product (SM) of Reaction 1f, 77 mL (0.55 mmole) of triethylamine (TEA) and 3.36 mg (0.0275 mmole) of 4-dimethylaminopyridine (DMAP) were added in 2 ml of methylene chloride (MC), and stirred, followed by stirring at room temperature for 30 minutes. 31.2 mL (0.358 mmole) of propionyl chloride was slowly added dropwise in the reaction solution and stirred at room temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC). The concentrate was purified with a flash column (eluent:ethyl acetate(EA):hexane(Hex)=1:3 (volume ratio) mixture) to obtain 51.2 mg of the target compound (yield: 63.7%).

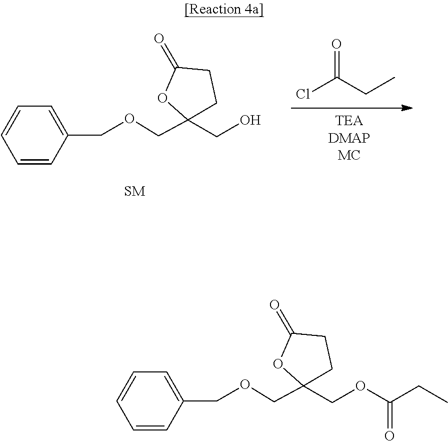

B. As shown in Reaction 4b below, 51.2 mg (0.175 mmole) of the product (SM) of Reaction 4a was dissolved in 1 ml of methylene chloride (MC). Then, after bubbling with nitrogen gas (N₂), it was cooled to −78° C. 0.53 ml (0.525 mmole) of boron trichloride (1M in MC, BCl₃) was slowly added dropwise in the coolant, and stirred at the same temperature for 2 hours. The reaction was confirmed by TLC (EA:Hex=1:2). When the reaction was completed, a saturated sodium hydrogen carbonate solution (Saturated NaHCO₃ soln.) was added to the reaction solution to quench the reaction, and the temperature was raised to room temperature. It was extracted with methylene chloride (MC) and concentrated. The concentrate was purified with a flash column (eluent:ethyl acetate(EA):hexane(Hex)=2:1 (volume ratio) mixture) to obtain 25.6 mg of the target compound (yield: 72.3%).

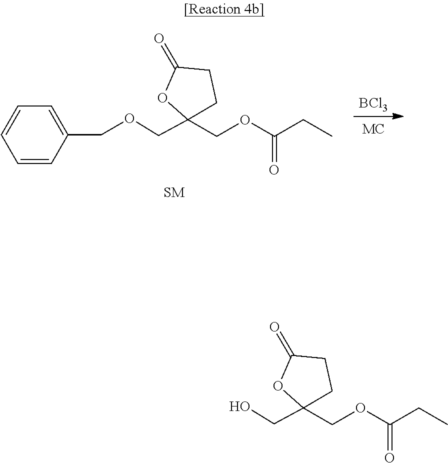 [SMILES: CCC(=O)OCC1(COCc2ccccc2)CCC(=O)O1 ; "BCl3" ; CCC(=O)OCC1(CO)CCC(=O)O1]

C. As shown in Reaction 4c below, 35.4 mg (0.126 mmole) of linoleic acid and 15.2 mL (0.1236 mmole) of pivaloyl chloride were added in 1 ml of methylene chloride (MC) and it was cooled to 0 to 5° C. 34.5 mL (0.2472 mmole) of triethylamine (TEA) was slowly added dropwise and stirred at the same temperature for 30 minutes. 25 mg (0.1236 mmole) of the product (SM) of Reaction 4b and 1.5 mg (0.0124 mmole) of 4-dimethylaminopyridine (DMAP) were added and stirred at room temperature for overnight. The reaction was confirmed by TLC (EA:Hex=1:2). When the reaction was completed, it was extracted with 0.1N-potassium hydroxide solution (KOH soln.)/methylene chloride (MC). Then, the extractive solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:3.5 (volume ratio) mixture) to obtain 29.9 mg of the target compound (yield: 52%).

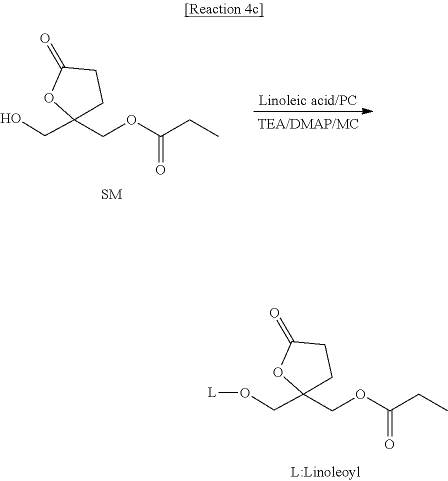

### [Example 5] Synthesis of Diacylglycerol Lactone Compound (EC-A116)

A. As shown in Reaction 5a below, 150 mg (0.635 mmole) of the product (SM) of Reaction 1f and 0.18 ml (1.27 mmole) of triethylamine (TEA) were added in 6.35 ml of methylene chloride (MC) and stirred at room temperature for 30 minutes. 86.2 mL (0.825 mmole) of butyryl chloride was slowly added dropwise in the reaction solution and stirred at room temperature for 3 hours. The reaction was confirmed by TLC (EA:Hex=1:2). When the reaction was completed, extraction was carried out with 0.1N-hydrochloric acid aqueous solution (c-HCl soln.)/methylene chloride (MC) and concentrated. The concentrate was purified with a flash column (eluent:ethyl acetate(EA):hexane(Hex)=1:3.5 (volume ratio) mixture) to obtain 106 mg of the target compound (yield: 54.5%).

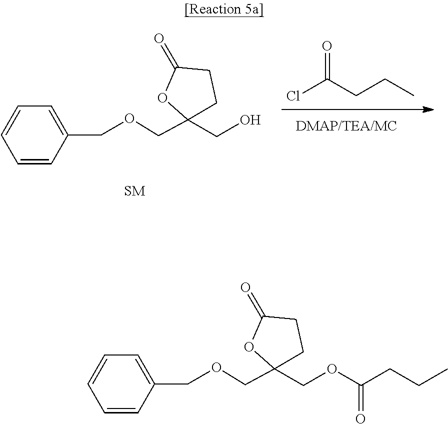

B. As shown in Reaction 5b below, 106 mg (0.346 mmole) of the product (SM) of Reaction 5a was dissolved in 1 ml of methylene chloride (MC). Then, after bubbling with nitrogen gas (N₂), it was cooled to −78° C. 1 ml (1.038 mmole) of boron trichloride (1M in MC, BCl3) was slowly added dropwise and stirred at the same temperature for 2 hours. The reaction was confirmed by TLC (EA:Hex=1:1). When the reaction was completed, a saturated sodium hydrogen carbonate solution (Saturated NaHCO₃aq. Soln.) was added to the reaction solution to quench the reaction and it was heated to room temperature. It was extracted with methylene chloride (MC). The concentrate was purified with a flash column (eluent:ethyl acetate(EA):hexane(Hex)=1:1 (volume ratio) mixture) to obtain 52.8 mg of the target compound (yield: 70.5%).

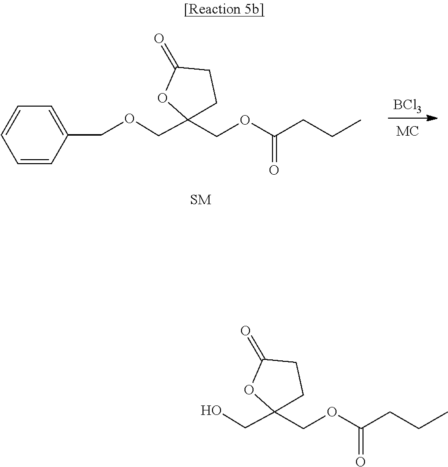

C. As shown in Reaction 5c below, 70 mg (0.249 mmole) of linoleic acid and 30 mL (0.24436 mmole) of pivaloyl chloride were added in 1 ml of methylene chloride (MC) and it was cooled to 0 to 5° C. 68 mL (0.488 mmole) of triethylamine (TEA) was slowly added dropwise and stirred at the same temperature for 30 minutes. 52.8 mg (0.244 mmole) of the product (SM) of Reaction 5b and 3 mg (0.0244 mmole) of 4-dimethylaminopyridine (DMAP) were added in the reaction solution at the same temperature and stirred at the room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent: ethyl acetate(EA):hexane(Hex)=1:3.5 (volume ratio) mixture) to obtain 78.4 mg of the target compound (yield: 67%).

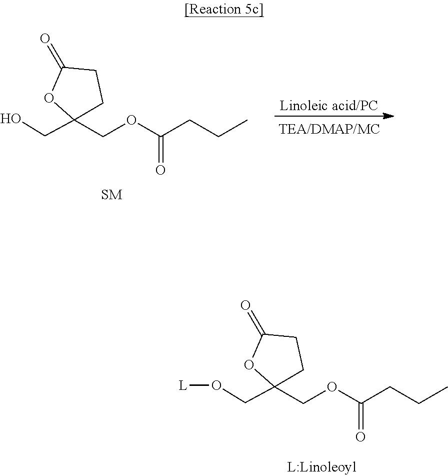

### [Example 6] Synthesis of Diacylglycerol Lactone Compound (EC-A117)

A. As shown in Reaction 6a below, 150 mg (0.635 mmole) of the product (SM) of Reaction 1f and 0.18 ml (1.27 mmole) of triehtlyamine (TEA) were added in 2 ml of methylene chloride (MC) and stirred. Then It was stirred at room temperature for 30 minutes. 0.1 ml (0.825 mmole) of valeroyl chloride was slowly added dropwise in the reaction solution and stirred at room temperature for 3 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3.5 (volume ratio) mixture) to obtain 89.1 mg of the target compound (yield: 43.8%).

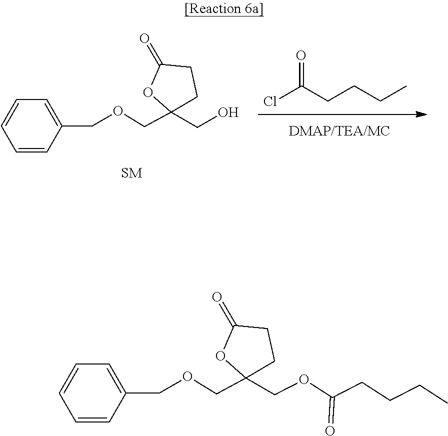

B. As shown in Reaction 6b below, 89.1 mg (0.278 mmole) of the product (SM) of Reaction 6a was dissolved in 1.4 ml of methylene chloride (MC). Then, after bubbling with nitrogen gas (N₂), it was cooled to −78° C. 0.83 ml (0.834 mmole) of boron trichloride (1M in MC, BCl₃) was slowly added dropwise. The reaction solution was stirred at the same temperature for 1 hour. The reaction was confirmed by TLC (EA:Hex=1:1). When the reaction was completed, a saturated sodium hydrogen carbonate solution (Saturated NaHCO₃aq. Soln.) was added to the reaction solution to quench the reaction and it was heated to room temperature. It was extracted with methylene chloride (MC) and then concentrated. The concentrate was purified with a flash column (eluent:ethyl acetate(EA):hexane(Hex)=1:1 (volume ratio) mixture) to obtain 46.8 mg of the target compound (yield: 73.1%).

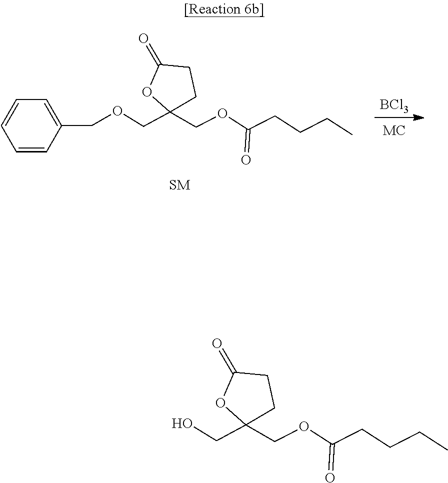 [SMILES: CCCCC(=O)OCC1(COCc2ccccc2)CCC(=O)O1 ; "BCl3" ; CCCCC(=O)OCC1(CO)CCC(=O)O1]

C. As shown in Reaction 6c below, 58.1 mg (0.207 mmole) of linoleic acid and 25 mL (0.203 mmole) of pivaloyl chloride was added in 1 ml of methylene chloride (MC) and it was cooled to 0 to 5° C. 57 mL (0.406 mmole) of triethylamine (TEA) was slowly added dropwise and stirred at the same temperature for 30 minutes. 46.8 mg (0.203 mmole) of the product (SM) of Reaction 6b and 2.5 mg (0.0203 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:3.5 (volume ratio) mixture) to obtain 42.5 mg of the target compound (yield: 42.5%).

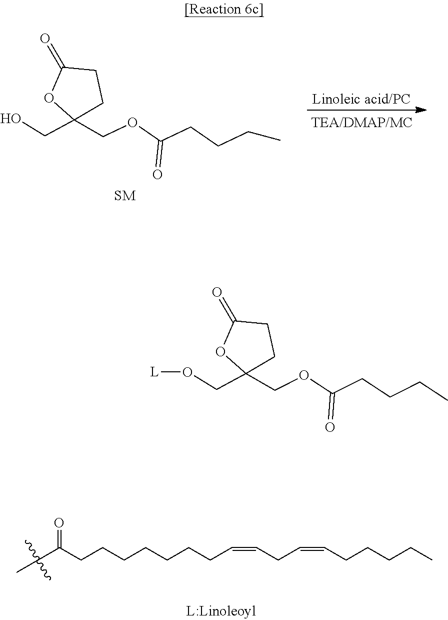

### [Example 7] Synthesis of Diacylglycerol Lactone Compound (EC-A118)

A. As shown in Reaction 7a below, 150 mg (0.635 mmole) of the product (SM) of Reaction 1f, 0.18 ml (1.27 mmole) of triethylamine (TEA) and 7.8 mg (0.063 mmole) of 4-dimethylaminopyridine (DMAP) were added in 2 ml of methylene chloride (MC) and stirred. Then, it was stirred at room temperature for 30 minutes. 0.113 mL (0.825 mmole) of hexanoyl chloride was slowly added dropwise. The reaction solution was stirred at room temperature for 3 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3.5 (volume ratio) mixture) to obtain 135.4 mg of the product (yield: 63.7%).

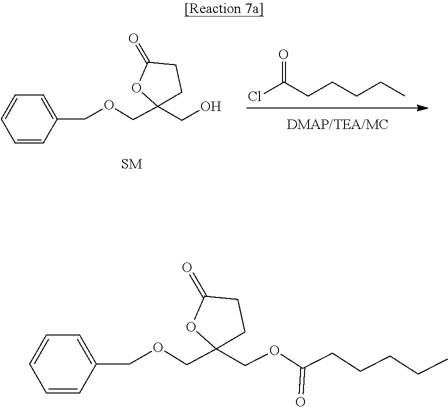

B. As shown in Reaction 7b below, 135.4 mg (0.405 mmole) of the product (SM) of Reaction 7a was dissolved in 2 ml of methylene chloride (MC). Then, after bubbling with nitrogen gas (N₂), it was cooled to −78° C. 1.2 ml (1.21 mmole) of boron trichloride (1M in MC, BCl₃) was slowly added dropwise. The reaction solution was stirred at the same temperature for 2 hours. The reaction was confirmed by TLC (EA:Hex=1:1). When the reaction was completed, a saturated sodium hydrogen carbonate solution (Saturated NaHCO₃aq. Soln.) was added to the reaction solution to quench the reaction, and it was heated to room temperature. It was extracted with methylene chloride (MC) and concentrated. The concentrate was purified with a flash column (eluent:ethyl acetate(EA):hexane(Hex)=1:1 (volume ratio) mixture) to obtain 70.5 mg mg of the product (yield: 71.3%).

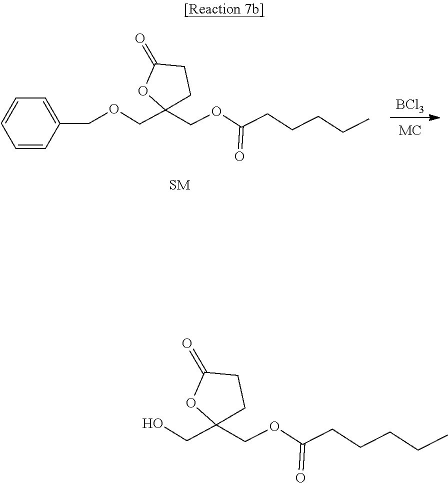 [SMILES: CCCCCC(=O)OCC1(COCc2ccccc2)CCC(=O)O1 ; "BCl3" ; CCCCCC(=O)OCC1(CO)CCC(=O)O1]

C. As shown in Reaction 7c below, 83 mg (0.294 mmole) of linoleic acid and 35.5 mL (0.2886 mmole) of pivaloyl chloride were added in 1 ml of methylene chloride (MC). Then, It was cooled to 0 to 5° C. 80 mL (0.5772 mmole) of triethylamine (TEA) was slowly added dropwise and stirred at the same temperature for 30 minutes. 70.5 mg (0.2886 mmole) of the product (SM) of Reaction 7b and 4.3 mg (0.035 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 20 mg of the target compound (yield: 13.7%).

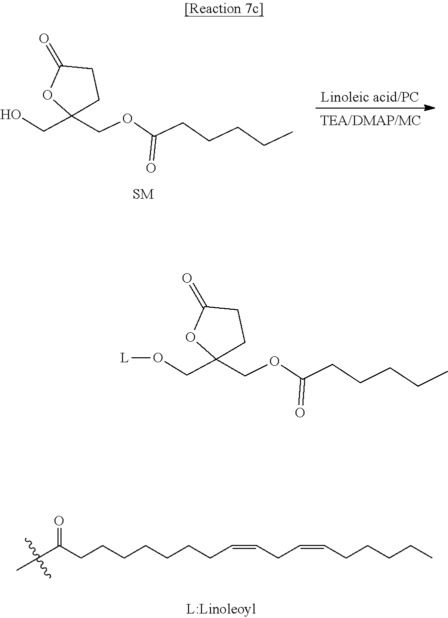

### [Example 8] Synthesis of Diacylglycerol Lactone Compound (EC-A119)

A. As shown in Reaction 8a below, 150 mg (0.635 mmole) of the product (SM) of Reaction 1f, 0.18 ml (1.27 mmole) of triethylamine (TEA) and 7.8 mg (0.063 mmole) of 4-dimethylaminopyridine (DMAP) were added in 3 ml of methylene chloride (MC) and stirred. Then, it was stirred at room temperature for 30 minutes. 0.13 ml (0.825 mmole) of heptanoyl chloride was slowly added dropwise and stirred at room temperature for 3 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3.5 (volume ratio) mixture) to obtain 126.1 mg of the product (yield: 57%).

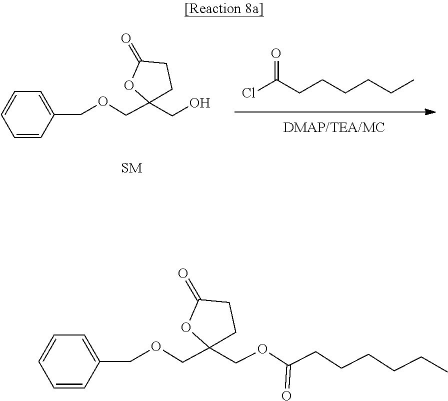

B. As shown in Reaction 8b below, 126 mg (0.362 mmole) of the product (SM) of Reaction 8a was dissolved in 2 ml of methylene chloride (MC). Then, after bubbling with nitrogen gas (N₂), it was cooled to −78° C. 1.1 ml (1.08 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and concentrated. The concentrate was purified with a flash column (eluent:ethyl acetate(EA):hexane(Hex)=1:1 (volume ratio) mixture) to obtain 77.8 mg of the product (yield: 83.2%).

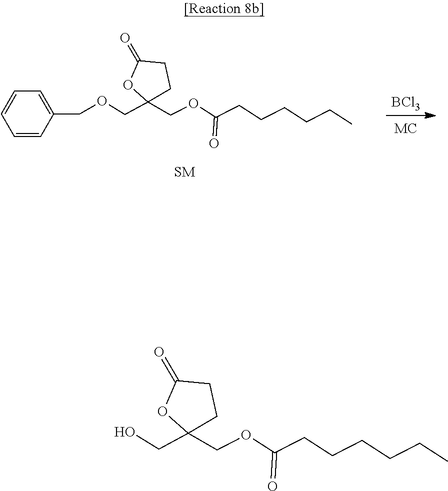 [SMILES: CCCCCCC(=O)OCC1(COCc2ccccc2)CCC(=O)O1 ; "BCl3" ; CCCCCCC(=O)OCC1(CO)CCC(=O)O1]

C. As shown in Reaction 8c below, 86.2 mg (0.3072 mmole) of linoleic acid and 37 mL (0.3012 mmole) of pivaloyl chloride were added in 1 ml of methylene chloride (MC). Then, It was cooled to 0 to 5° C. 84 mL (0.6024 mmole) of triethylamine (TEA) was slowly added dropwise and stirred at the same temperature for 30 minutes. 77.8 mg (0.3012 mmole) of the product (SM) of Reaction 8b and 3.7 mg (0.0301 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:3.5 (volume ratio) mixture) to obtain 75.1 mg of the target compound (yield: 47.9%).

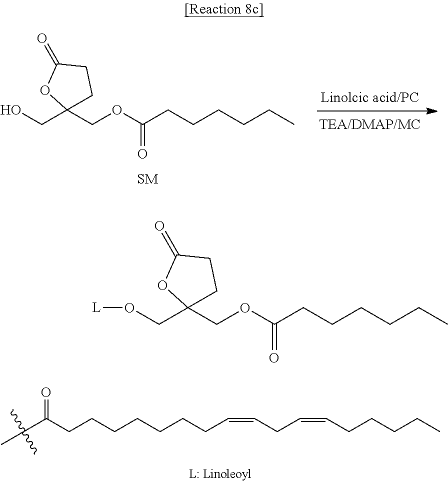

### [Example 9] Synthesis of Diacylglycerol Lactone Compound (EC-A120)

A. As shown in Reaction 9a below, 150 mg (0.635 mmole) of the product (SM) of Reaction 1f, 0.18 ml (1.27 mmole) of triethylamine (TEA) and 7.8 mg (0.063 mmole) of 4-dimethylaminopyridine (DMAP) were added in 2 ml of methylene chloride (MC) and stirred. Then, it was stirred at room temperature for 30 minutes. 0.16 ml (0.825 mmole) of nonanoyl chloride was slowly added dropwise. The reaction solution was stirred at room temperature for 3 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3.5 (volume ratio) mixture) to obtain 154 mg of the target compound (yield: 64%).

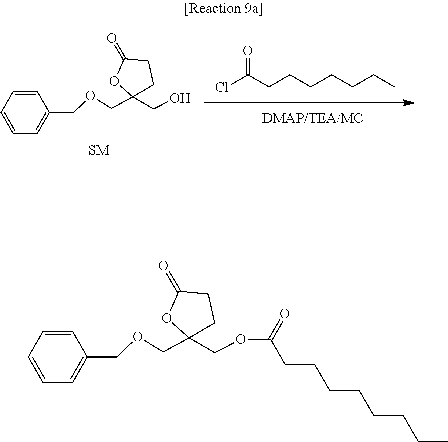

B. As shown in Reaction 9b below, 154 mg (0.41 mmole) of the product (SM) of Reaction 9a was dissolved in 2 ml of methylene chloride (MC). Then, after bubbling with nitrogen gas (N₂), it was cooled to −78° C. 1.21 ml (1.227 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:1 (volume ratio) mixture) to obtain 99.7 mg of the target compound (yield: 85%).

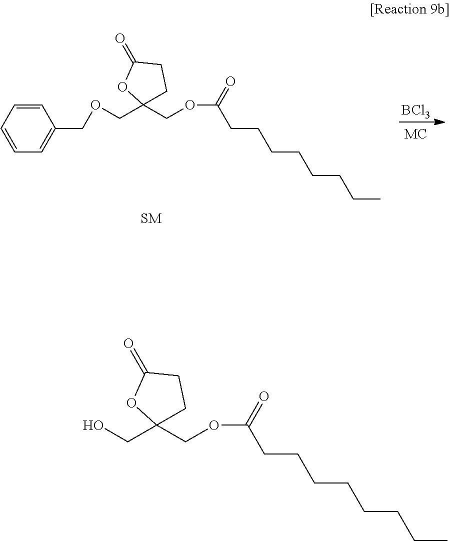 [SMILES: CCCCCCCCC(=O)OCC1(COCc2ccccc2)CCC(=O)O1 ; "BCl3" ; CCCCCCCCC(=O)OCC1(CO)CCC(=O)O1]

C. As shown in Reaction 9c below, 100 mg (0.355 mmole) of linoleic acid and 43 mL (0.355 mmole) of pivaloyl chloride were added in 1 ml of methylene chloride (MC). Then, it was cooled to 0 to 5° C. 97 mL (0.696 mmole) of triethylamine (TEA) was slowly added dropwise. The reaction solution was stirred at the same temperature for 30 minutes. 99.7 mg (0.348 mmole) of the product (SM) of Reaction 9b and 4.3 mg (0.035 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:3.5 (volume ratio) mixture) to obtain 87.1 mg of the target compound (yield: 45.6%).

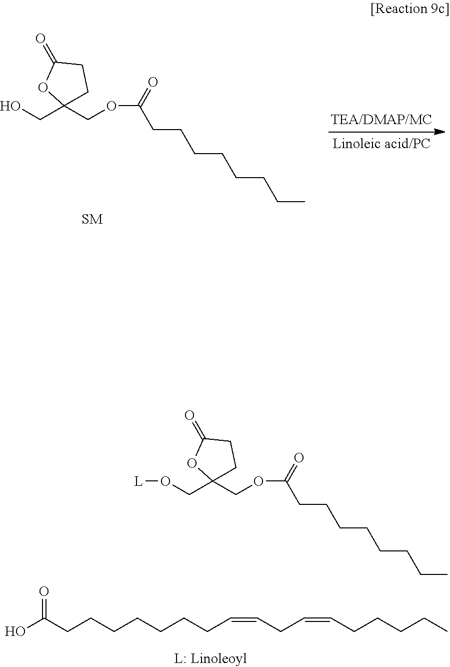

### [Example 10] Synthesis of Diacylglycerol Lactone Compound (EC-A121)

A. As shown in Reaction 10a below, 165.264 mg (0.825 mmole) of lauric acid and 43 mL (0.825 mmole) of pivaloyl chloride were added in 2 ml of methylene chloride (MC). Then, It was cooled to 0° C. 0.27 ml (3 mmole) of triethylamine (TEA) was slowly added dropwise. The reaction solution was stirred at the same temperature for 30 minutes. 150 mg (0.635 mmole) of the product (SM) of Reaction 1f and 7.8 mg (0.0635 mmole) of 4-dimethylaminopyridine (DMAP) were added at the same temperature and stirred at room temperature for 2 hours. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 159.1 mg of the target compound (yield: 59.8%).

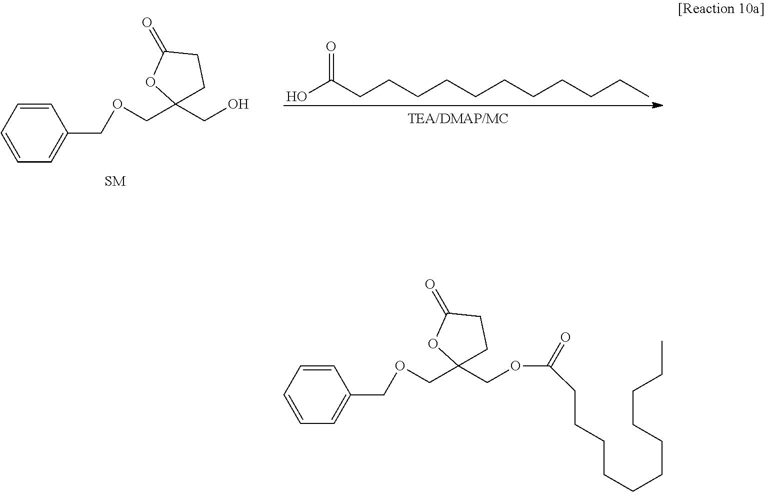 [SMILES: O=C1CCC(CO)(COCc2ccccc2)O1 ; CCCCCCCCCCCC(=O)O ; CCCCCCCCCCCC(=O)OCC1(COCc2ccccc2)CCC(=O)O1]

B. As shown in Reaction 10b below, 159 mg (0.3798 mmole) of the product (SM) of Reaction 10a was dissolved in 1.9 ml of methylene chloride (MC). Then, after bubbling with nitrogen gas (N₂), it was cooled to −78° C. 1.14 ml (1.14 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, 0.1N-hydrochloric acid solution (c-HCl soln.)/methylene chloride (MC) and then concentrated. It was obtained 113.6 mg of the target compound (yield: 91.1%).

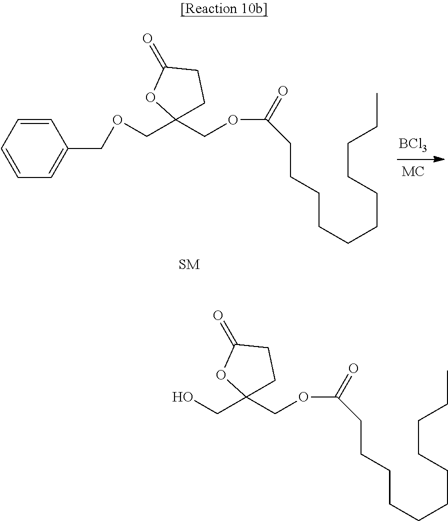

C. As shown in Reaction 10c below, 99 mg (0.3528 mmole) of linoleic acid and 42.56 mL (0.3459 mmole) of pivaloyl chloride were added in 1 ml of methylene chloride (MC). Then, It was cooled to 0° C. 0.15 ml (1.0377 mmole) of triethylamine (TEA) was slowly added dropwise. The reaction solution was stirred at the same temperature for 30 minutes. 113.6 mg (0.3459 mmole) of the product (SM) of Reaction 10b and 4 mg (0.0346 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/Methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:5 (volume ratio) mixture) to obtain 128 mg of the target compound (yield: 62.6%).

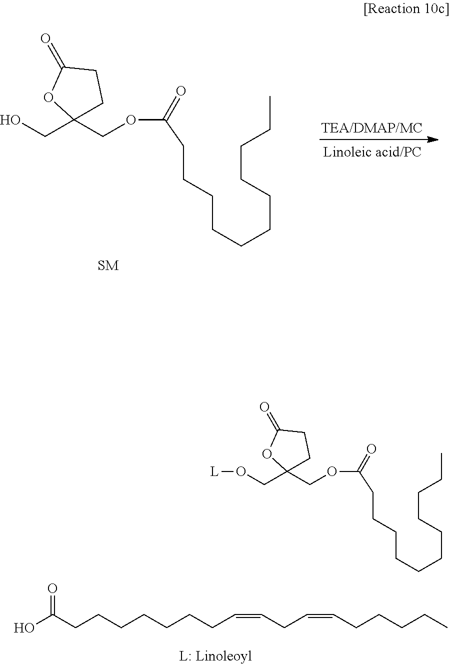

### [Example 11] Synthesis of Diacylglycerol Lactone Compound (EC-A122)

A. As shown in Reaction 10a below, 188.405 mg (0.825 mmole) of myristic acid and 43 mL (0.825 mmole) of pivaloyl chloride were added in 2 ml of methylene chloride (MC). Then, It was cooled to 0° C. 0.27 ml (3 mmole) of triethylamine (TEA) was slowly added dropwise. The reaction solution was stirred at the same temperature for 30 minutes. 150 mg (0.635 mmole) of the product (SM) of Reaction 1f and 7.8 mg (0.0635 mmole) of 4-dimethylaminopyridine (DMAP) were added at the same temperature and stirred at room temperature for 2 hours. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 190.7 mg of the target compound (yield: 67.2%).

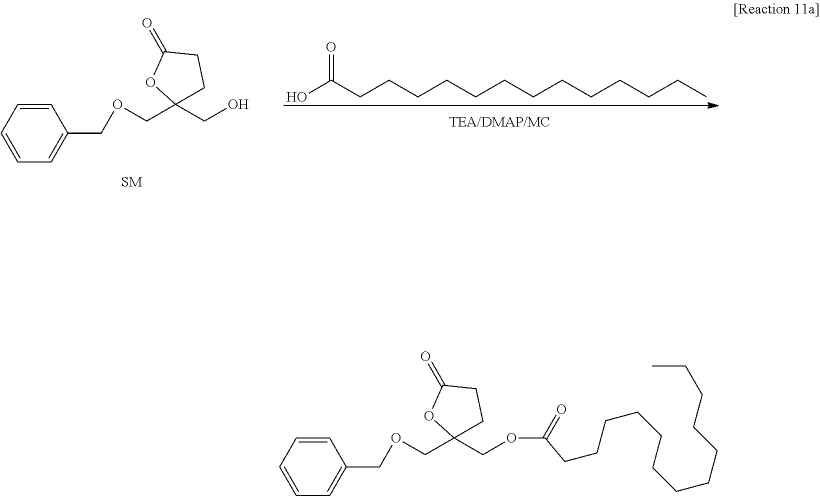 [SMILES: O=C1CCC(CO)(COCc2ccccc2)O1 ; CCCCCCCCCCCCCC(=O)O ; CCCCCCCCCCCCCC(=O)OCC1(COCc2ccccc2)CCC(=O)O1]

B. As shown in Reaction 11b below, 190.7 mg (0.427 mmole) of the product (SM) of Reaction 10a was dissolved in 2 ml of methylene chloride (MC). Then, after bubbling with nitrogen gas (N₂), it was cooled to −78° C. 1.28 ml (1.28 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. It was obtained 111.2 mg of the target compound (yield: 73%).

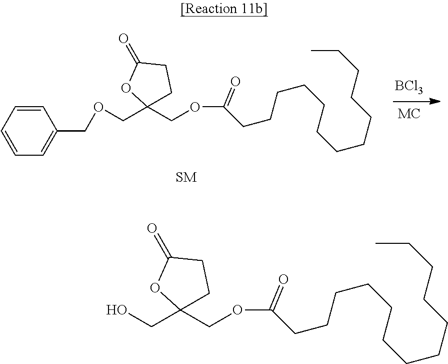

C. As shown in Reaction 11c below, 89 mg (0.3176 mmole) of linoleic acid and 38 mL (0.3012 mmole) of pivaloyl chloride were added in 1 ml of methylene chloride (MC). Then, it was cooled to 0° C. 94.5 mL (0.9341 mmole) of triethylamine (TEA) was slowly added dropwise. The reaction solution was stirred at the same temperature for 30 minutes. 111 mg (0.3113 mmole) of the product (SM) of Reaction 10b and 3.8 mg (0.031 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:5 (volume ratio) mixture) to obtain 104.5 mg of the target compound (yield: 54.2%).

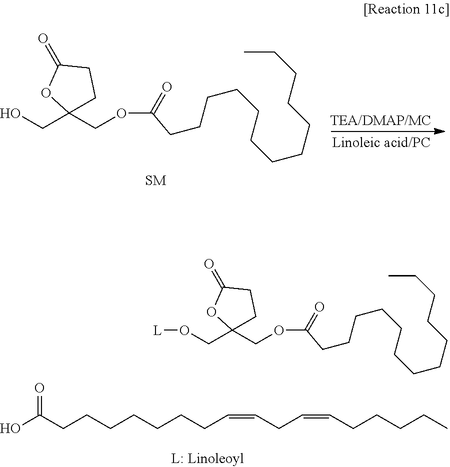

### [Example 12] Synthesis of Diacylglycerol Lactone Compound (EC-A123)

A. As shown in Reaction 12a below, 257.84 mg (0.825 mmole) of arachidonic acid and 43 mL (0.825 mmole) of pivaloyl chloride were added in 2 ml of methylene chloride (MC). Then, it was cooled to 0° C. 0.27 ml (3 mmole) of triethylamine (TEA) was slowly added dropwise. The reaction solution was stirred at the same temperature for 30 minutes. 150 mg (0.635 mmole) of the product (SM) of Reaction 1f and 7.8 mg (0.0635 mmole) of 4-dimethylaminopyridine (DMAP) were added at the same temperature and stirred at room temperature for 2 hours. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 217.6 mg of the target compound (yield: 64.6%).

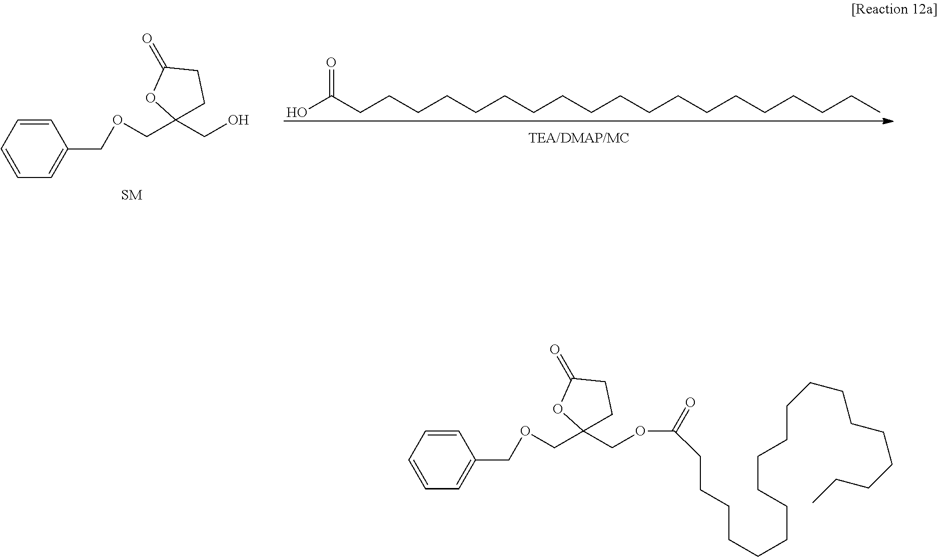 [SMILES: O=C1CCC(CO)(COCc2ccccc2)O1 ; CCCCCCCCCCCCCCCCCCCC(=O)O ; CCCCCCCCCCCCCCCCCCCC(=O)OCC1(COCc2ccccc2)CCC(=O)O1]

B. As shown in Reaction 12b below, 217.6 mg (0.498 mmole) of the product (SM) of Reaction 12a was dissolved in 2 ml of methylene chloride (MC). Then, after bubbling with nitrogen gas (N₂), it was cooled to −78° C. 1.23 ml (1.23 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and then concentrated. It was obtained 153 mg of the target compound (yield: 84.7%).

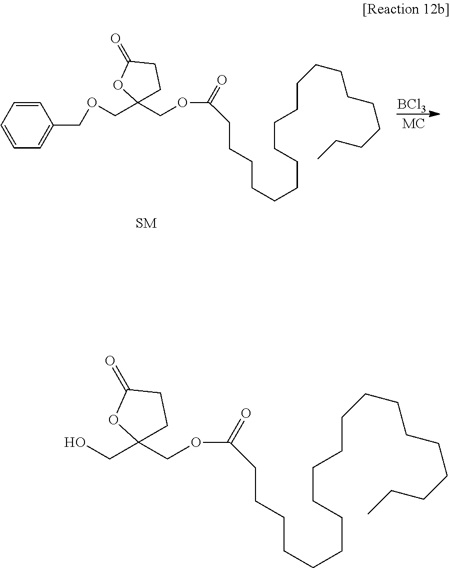

C. As shown in Reaction 12c below, 99.3 mg (0.3542 mmole) of linoleic acid and 42.7 mL (0.3472 mmole) of pivaloyl chloride were added in 1 ml of methylene chloride (MC). Then, it was cooled to 0° C. 0.15 ml (1.0416 mmole) of triethylamine (TEA) was slowly added dropwise. The reaction solution was stirred at the same temperature for 30 minutes. 153 mg (0.3472 mmole) of the product (SM) of Reaction 12b and 4.2 mg (0.035 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:5 (volume ratio) mixture) to obtain 113.2 mg of the target compound (yield: 46.3%).

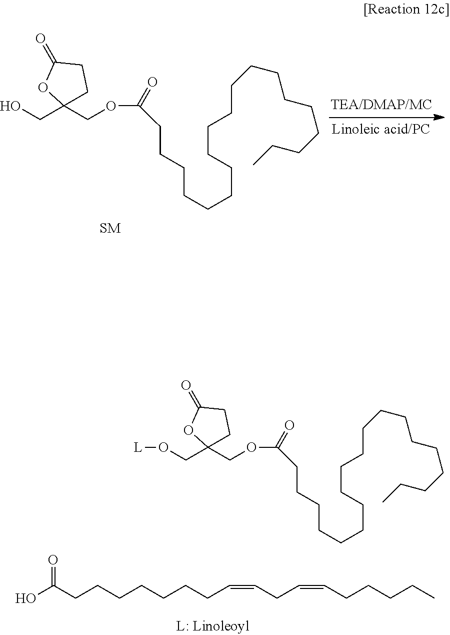

### [Example 13] Synthesis of Diacylglycerol Lactone Compound (EC-A124)

A. As shown in Reaction 13a below, 150 mg (0.635 mmole) of the product (SM) of Reaction 1f was dissolved in 2 ml of methylene chloride (MC). Then, 0.18 ml of triethylamine (TEA) and 7.8 mg of 4-dimethylaminopyridine (DMAP) were added and stirred. Then, it was stirred at room temperature for 30 minutes. 87 mL (0.825 mmole) of isobutyryl chloride was slowly added dropwise in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:4 (volume ratio) mixture) to obtain 111.7 mg of the product (yield: 57.4%).

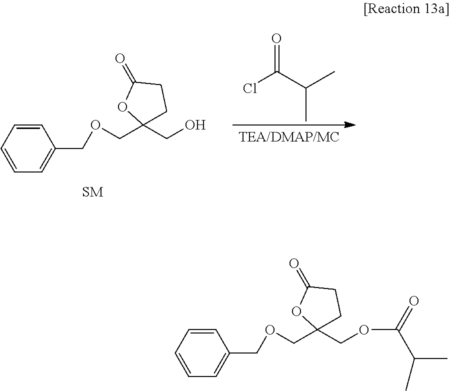

B. As shown in Reaction 13b below, 111.7 mg (0.3646 mmole) of the product (SM) of Reaction 13a was dissolved in 2 ml of methylene chloride (MC). Then, after bubbling with nitrogen gas (N₂), it was cooled to −78° C. 1.1 ml (1.09 mmole) of boron trichloride (1M in MC, BCl₃) was slowly added dropwise. The reaction solution was stirred at the same temperature for 2 hours. The reaction was confirmed by TLC (EA:Hex=1:1). When the reaction was completed, extraction was carried out with 0.1N-hydrochloric acid aqueous solution (c-HCl soln.)/methylene chloride (MC) and concentrated. The concentrate was purified with a flash column (eluent:ethyl acetate(EA):hexane(Hex)=1:1 (volume ratio) mixture) to obtain 61.7 mg of the product (yield: 78.3%).

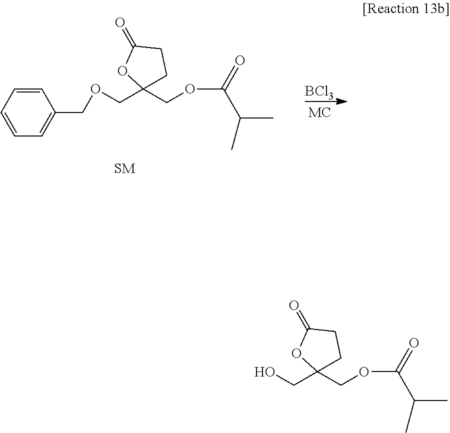 [SMILES: CC(C)C(=O)OCC1(COCc2ccccc2)CCC(=O)O1 ; "BCl3" ; CC(C)C(=O)OCC1(CO)CCC(=O)O1]

C. As shown in Reaction 13c below, 81.6 mg (0.291 mmole) of linoleic acid and 37 mL (0.3012 mmole) of pivaloyl chloride were added in 1 ml of methylene chloride (MC). Then, it was cooled to 0° C. 0.12 ml (0.8559 mmole) of triethylamine (TEA) was slowly added dropwise and stirred at the same temperature for 30 minutes. 61.7 mg (0.2853 mmole) of the product (SM) of Reaction 13b and 3.5 mg (0.029 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:5 (volume ratio) mixture) to obtain 110.91 mg of the target compound (yield: 81.2%).

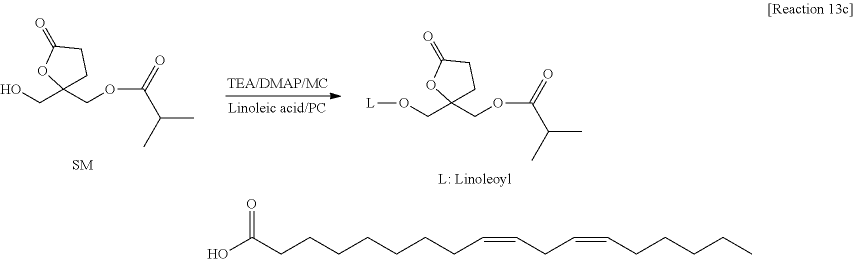

### [Example 14] Synthesis of Diacylglycerol Lactone Compound (EC-A125)

A. As shown in Reaction 14a below, 150 mg (0.635 mmole) of the product (SM) of Reaction 1f was dissolved in 2 ml of methylene chloride (MC). Then, 0.18 ml (1.27 mmole) of triethylamine (TEA) and 7.8 mg (0.064 mmole) of 4-dimethylaminopyridine (DMAP) were added and stirred. Then, it was stirred at room temperature for 30 minutes. 0.1 ml (0.825 mmole) of pivaloyl chloride was slowly added dropwise and stirred at room temperature for 2 hours. The reaction was confirmed by TLC (EA:Hex=1:1.5). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:4 (volume ratio) mixture) to obtain 117.5 mg of the product (yield: 57.7%).

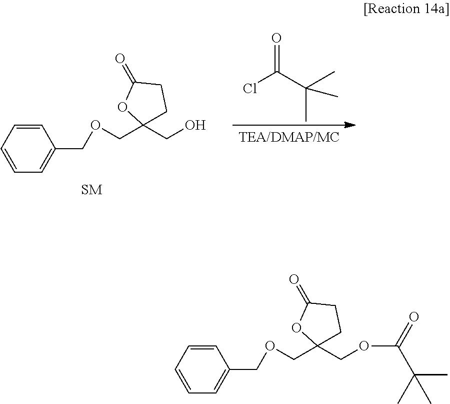

B. As shown in Reaction 14b below, 117 mg (0.3652 mmole) of the product (SM) of Reaction 14a was dissolved in 1.83 ml of methylene chloride (MC). Then, after bubbling with nitrogen gas (N₂), it was cooled to −78° C. 1.1 ml (1.09 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and then concentrated. It was obtained 73.4 mg of the target compound (yield: 87.3%).

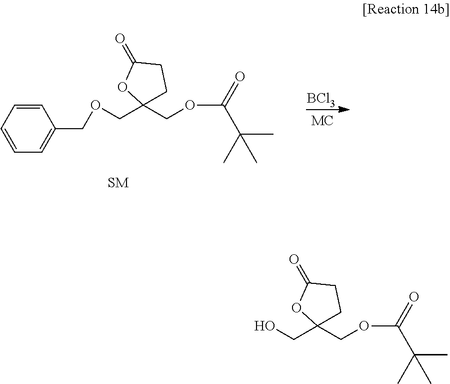 [SMILES: CC(C)(C)C(=O)OCC1(COCc2ccccc2)CCC(=O)O1 ; "BCl3" ; CC(C)(C)C(=O)OCC1(CO)CCC(=O)O1]

C. As shown in Reaction 14c below, 91.2 mg (0.3251 mmole) of linoleic acid and 40 mL (0.31877 mmole) of pivaloyl chloride were added in 1 ml of methylene chloride (MC). Then, it was cooled to 0° C. 0.13 ml (0.9563 mmole) of triethylamine (TEA) was slowly added dropwise. It was stirred at the same temperature for 30 minutes. 73.4 mg (0.31877 mmole) of the product (SM) of Reaction 14b and 4 mg (0.032 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:5 (volume ratio) mixture) to obtain 73.6 mg of the target compound (yield: 46.9%).

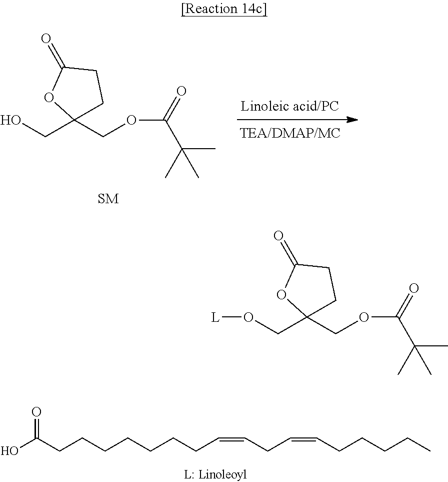

### [Example 15] Synthesis of Diacylglycerol Lactone Compound (EC-A126)

A. As shown in Reaction 15a below, 150 mg (0.635 mmole) of the product (SM) of Reaction 1f was dissolved in 2 ml of methylene chloride (MC). Then, 0.18 ml (1.27 mmole) of triethylamine (TEA) and 7.8 mg (0.064 mmole) of 4-dimethylaminopyridine (DMAP) were added and stirred. It was stirred at 0° C. for 30 minutes. 71 mL (0.825 mmole) of 2-methylbutyryl chloride was added in the reaction solution and stirred at room temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and then concentrated. It was obtained 102.4 mg of the target compound (yield: 50.3%).

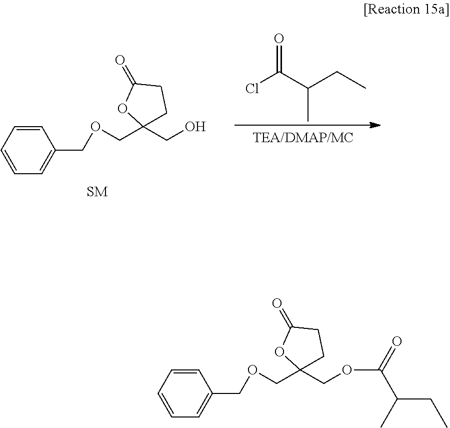

B. As shown in Reaction 15b below, 102 mg (0.31842 mmole) of the product (SM) of Reaction 15a was dissolved in 1.6 ml of methylene chloride (MC). Then, after bubbling with nitrogen gas (N₂), it was cooled to −78° C. 1.0 ml (0.955 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and then concentrated. It was obtained 58.5 mg of the target compound (yield: 79.8%).

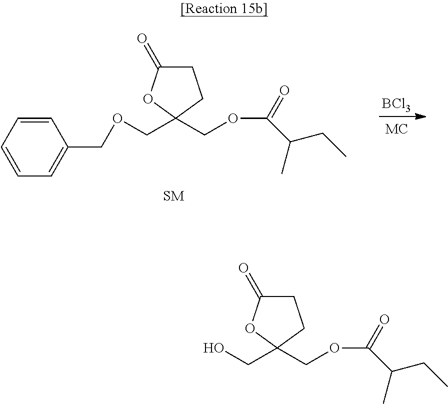 [SMILES: CCC(C)C(=O)OCC1(COCc2ccccc2)CCC(=O)O1 ; "BCl3" ; CCC(C)C(=O)OCC1(CO)CCC(=O)O1]

C. As shown in Reaction 15c below, 72.7 mg (0.259 mmole) of linoleic acid and 31 mL (0.254 mmole) of pivaloyl chloride were added in 1 ml of methylene chloride (MC). Then, it was cooled to 0° C. 0.11 ml (0.762 mmole) of triethylamine (TEA) was slowly added dropwise. It was stirred at the same temperature for 30 minutes. 58.5 mg (0.254 mmole) of the product (SM) of Reaction 15b and 3.1 mg (0.0254 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:5 (volume ratio) mixture) to obtain 64.7 mg of the target compound (yield: 51.7%).

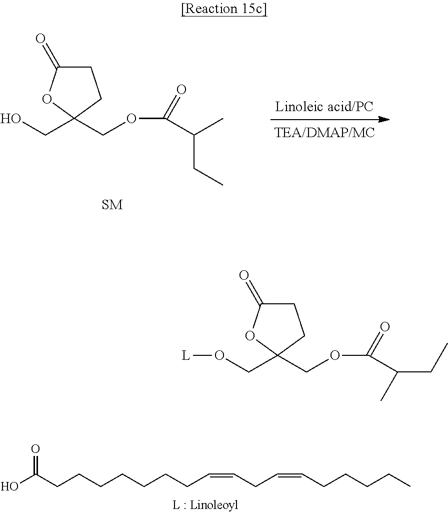

### [Example 16] Synthesis of Diacylglycerol Lactone Compound (EC-A127)

A. As shown in Reaction 16a below, 150 mg (0.635 mmole) of the product (SM) of Reaction 1f was dissolved in 2 ml of methylene chloride (MC). Then, 0.18 ml (1.27 mmole) of triethylamine (TEA) and 7.8 mg (0.064 mmole) of 4-dimethylaminopyridine (DMAP) were added and stirred. Then, it was stirred at 0° C. for 30 minutes. 75 mL (0.825 mmole) of cyclopropanecarbonyl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3.5 (volume ratio) mixture) to obtain 123.3 mg of the product (yield: 63.8%).

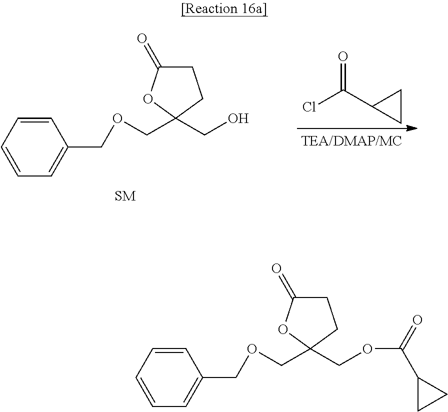 [SMILES: O=C1CCC(CO)(COCc2ccccc2)O1 ; O=C(Cl)C1CC1 ; O=C1CCC(COCc2ccccc2)(COC(=O)C2CC2)O1]

B. As shown in Reaction 15b below, 123.3 mg (0.405 mmole) of the product (SM) of Reaction 15a was dissolved in 1.4 ml of methylene chloride (MC). Then, after bubbling with nitrogen gas (N₂), it was cooled to −78° C. 1.2 ml (1.215 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and concentrated. The concentrate was purified with a flash column (eluent:ethyl acetate(EA):hexane(Hex)=1:1 (volume ratio) mixture) to obtain 50 mg of the product (yield: 57.6%).

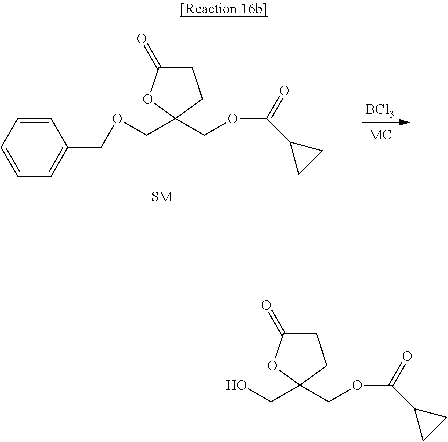 [SMILES: O=C1CCC(COCc2ccccc2)(COC(=O)C2CC2)O1 ; "BCl3" ; O=C1CCC(CO)(COC(=O)C2CC2)O1]

C. As shown in Reaction 16c below, 66.76 mg (0.238 mmole) of linoleic acid and 29 mL (0.2334 mmole) of pivaloyl chloride were added in 1 ml of methylene chloride (MC). Then, it was cooled to 0° C. 0.1 ml (0.7002 mmole) of triethylamine (TEA) was slowly added dropwise. The reaction solution was stirred at the same temperature for 30 minutes. 50 mg (0.2334 mmole) of the product (SM) of Reaction 16b and 2.85 mg (0.023 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 29.70 mg of the target compound (yield: 62.3%).

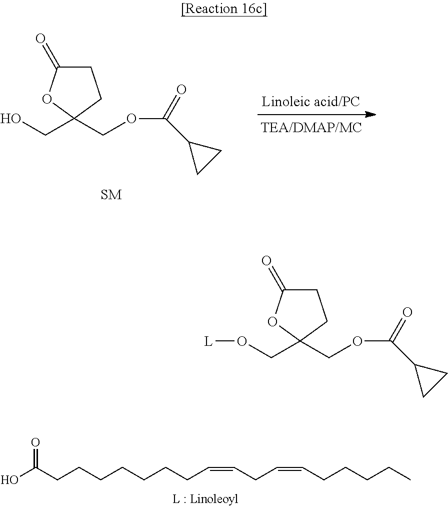

### [Example 17] Synthesis of Diacylglycerol Lactone Compound (EC-A128)

A. As shown in Reaction 17a below, 150 mg (0.635 mmole) of the product (SM) of Reaction 1f was dissolved in 2 ml of methylene chloride (MC). Then, 0.18 ml (1.27 mmole) of triethylamine (TEA) and 7.8 mg (0.064 mmole) of 4-dimethylaminopyridine (DMAP) were added and stirred. Then, it was stirred at 0° C. for 30 minutes. 0.11 ml (0.825 mmole) of cyclohexanecarbonyl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3.5 (volume ratio) mixture) to obtain 140.9 mg of the product (yield: 64%).

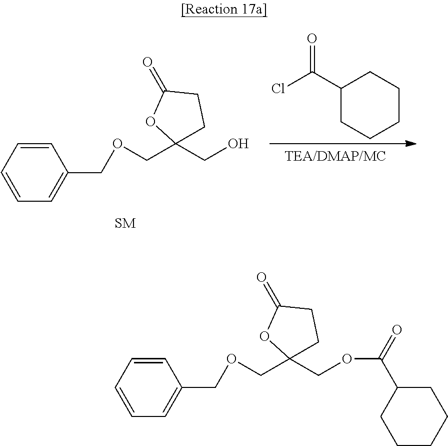 [SMILES: O=C1CCC(CO)(COCc2ccccc2)O1 ; O=C(Cl)C1CCCCC1 ; O=C1CCC(COCc2ccccc2)(COC(=O)C2CCCCC2)O1]

B. As shown in Reaction 17b below, 140.9 mg (0.4067 mmole) of the product (SM) of Reaction 17a was dissolved in 1.4 ml of methylene chloride (MC). Then, after bubbling with nitrogen gas (N₂), it was cooled to −78° C. 1.2 ml (1.22 mmole) of boron trichloride (1M in MC, BCl₃) was slowly added dropwise. The reaction solution was stirred at the same temperature for 2 hours. The reaction was confirmed by TLC (EA:Hex=1:2). When the reaction was completed, extraction was carried out with 0.1N-hydrochloric acid aqueous solution (c-HCl soln.)/methylene chloride (MC) and then concentrated. The concentrate was purified with a flash column (eluent:ethyl acetate(EA):hexane(Hex)=1:1 (volume ratio) mixture) to obtain 95.1 mg of the product (yield: 91.2%).

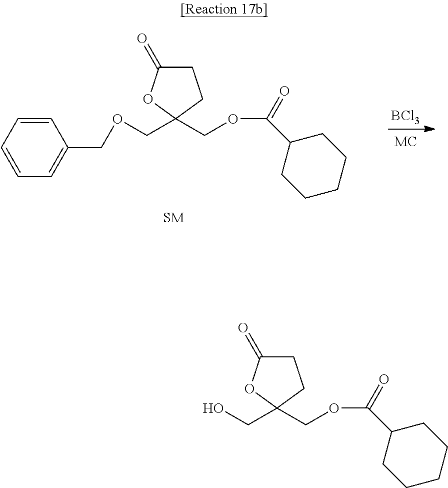 [SMILES: O=C1CCC(COCc2ccccc2)(COC(=O)C2CCCCC2)O1 ; "BCl3" ; O=C1CCC(CO)(COC(=O)C2CCCCC2)O1]

C. As shown in Reaction 17c below, 106.1 mg (0.378 mmole) of linoleic acid and 45.6 mL (0.371 mmole) of pivaloyl chloride were added in 1 ml of methylene chloride (MC). Then, it was cooled to 0° C. 0.15 ml (1.113 mmole) of triethylamine (TEA) was slowly added dropwise. The reaction solution was stirred at the same temperature for 30 minutes. 95.1 mg (0.371 mmole) of the product (SM) of Reaction 17b and 4.5 mg (0.037 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 126.4 mg of the target compound (yield: 65.7%).

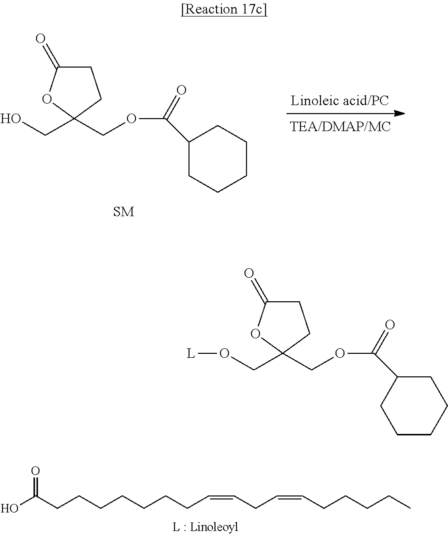

### [Example 18] Synthesis of Diacylglycerol Lactone Compound (EC-A130)

A. As shown in Reaction 18a below, 5.88 g (22.945 mmole) of palmitic acid and 2.88 ml (22.945 mmole) of pivaloyl chloride were added in 1 ml of methylene chloride (MC). Then, it was cooled to 0° C. 7.4 ml (52.95 mmole) of triethylamine (TEA) was slowly added dropwise. The reaction solution was stirred at the same temperature for 30 minutes. 1.47 g (17.65 mmole) of the product (SM) of Reaction 1f and 216 mg (1.765 mmole) of 4-dimethylaminopyridine (DMAP) were added at the same temperature and stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4.5 (volume ratio) mixture) to obtain 1.9 g of the target compound (yield: 22.6%).

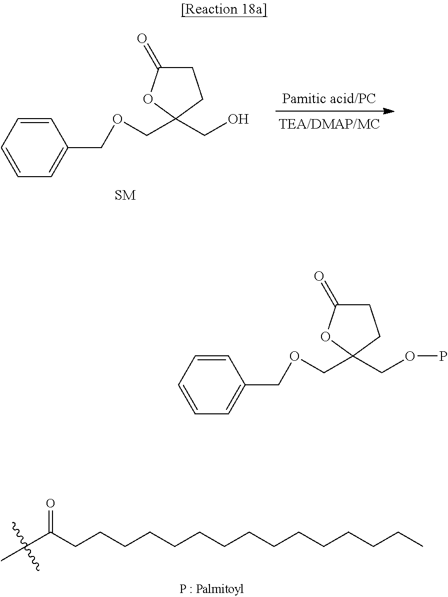

B. As shown in Reaction 18b below, 1.9 g (4.003 mmole) of the product (SM) of Reaction 18a was dissolved in 20 ml of methylene chloride (MC). Then, after bubbling with nitrogen gas (N₂), it was cooled to −78° C. 12 ml (12.01 mmole) of boron trichloride (1M in MC, BCl₃) was slowly added dropwise. The reaction solution was stirred at the same temperature for 2 hours. The reaction was confirmed by TLC (EA:Hex=1:1). When the reaction was completed, extraction was carried out with 0.1N-hydrochloric acid aqueous solution (c-HCl soln.)/methylene chloride (MC) and then concentrated. The concentrate was purified with a flash column (eluent:ethyl acetate(EA):hexane(Hex)=1:1.5 (volume ratio) mixture) to obtain 1.287 g of the target compound (yield: 83.6%).

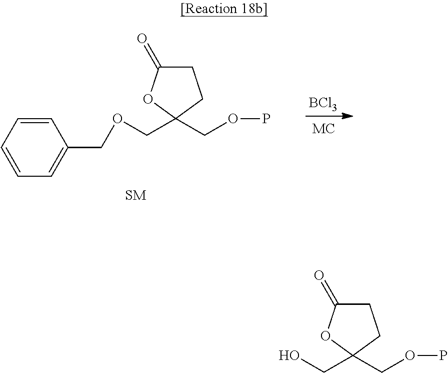

C. As shown in Reaction 18c below, 100 mg (0.26 mmole) of the product (SM) of Reaction 18b was dissolved in 1 ml of methylene chloride (MC). Then, 72.5 mL (0.52 mmole) of triethylamine (TEA) and 3.17 mg (0.026 mmole) of 4-dimethylaminopyridine (DMAP) were added and stirred. Then it was stirred at 0° C. for 30 minutes. 35.3 mL (0.338 mmole) of butyryl chloride was added in the reaction solution. it was stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:3 (volume ratio) mixture) to obtain 86.8 mg of the target compound (yield: 73.4%).

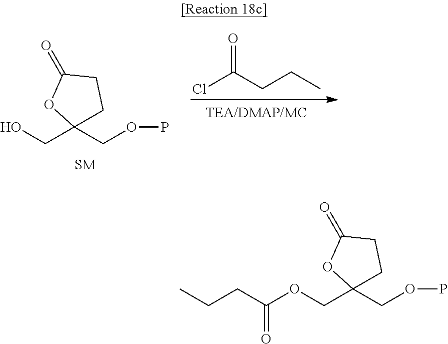

### [Example 19] Synthesis of Diacylglycerol Lactone Compound (EC-A131)

As shown in Reaction 19a below, 100 mg (0.26 mmole) of the product (SM) of Reaction 18b was dissolved in 1 ml of methylene chloride (MC). Then, 72.5 mL (0.52 mmole) of triethylamine (TEA) and 3.17 mg (0.026 mmole) of 4-dimethylaminopyridine (DMAP) were added and stirred. Then, it was stirred at 0° C. for 30 minutes. 36 mL (0.338 mmole) of isobutyryl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:4 (volume ratio) mixture) to obtain 86.8 mg of the product (yield: 73.4%).

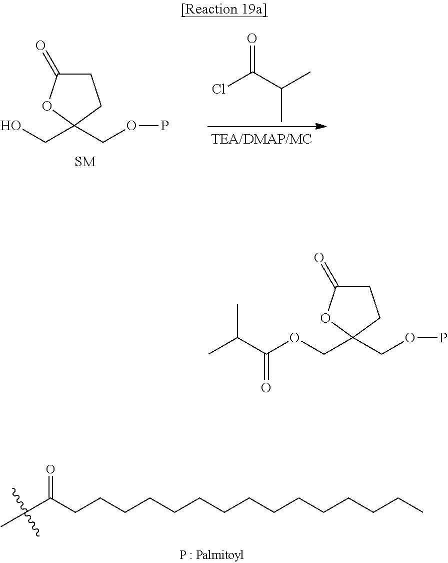

### [Example 20] Synthesis of Diacylglycerol Lactone Compound (EC-A132)

As shown in Reaction 20a below, 100 mg (0.26 mmole) of the product (SM) of Reaction 18b was dissolved in 1 ml of methylene chloride (MC). Then, 72.5 mL (0.52 mmole) of triethylamine (TEA) and 3.17 mg (0.026 mmole) of 4-dimethylaminopyridine (DMAP) were added and stirred. Then, it was stirred at 0° C. for 30 minutes. 41.6 mL (0.338 mmole) of pivaloyl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:4 (volume ratio) mixture) to obtain 84.9 mg of the product (yield: 69.7%).

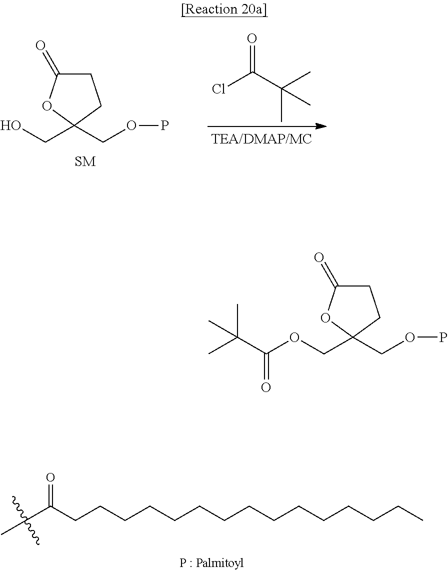

### [Example 21] Synthesis of Diacylglycerol Lactone Compound (EC-A133)

As shown in Reaction 21a below, 100 mg (0.26 mmole) of the product (SM) of Reaction 18b was dissolved in 1 ml of methylene chloride (MC). Then, 0.1 ml (0.78 mmole) of triethylamine (TEA) and 3.17 mg (0.026 mmole) of 4-dimethylaminopyridine (DMAP) were added and stirred. Then, it was stirred at 0° C. for 30 minutes. 29 mL (0.338 mmole) of 2-methylbutyryl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:4 (volume ratio) mixture) to obtain 87.6 mg of the product (yield: 71.9%).

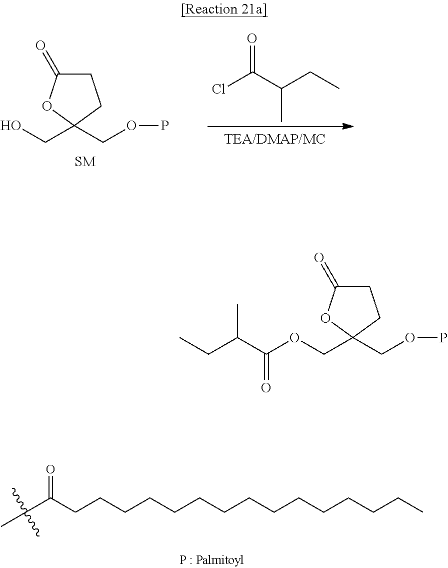

### [Example 22] Synthesis of Diacylglycerol Lactone Compound (EC-A134)

As shown in Reaction 22a below, 100 mg (0.26 mmole) of the product (SM) of Reaction 18b was dissolved in 1 ml of methylene chloride (MC). Then, 72.5 mL (0.52 mmole) of triethylamine (TEA) and 3.17 mg (0.026 mmole) of 4-dimethylaminopyridine (DMAP) were added and stirred. Then, it was stirred at 0° C. for 30 minutes. 30.7 mL (0.338 mmole) of cyclopropanecarbonyl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate (EA):hexane(Hex)=1:4 (volume ratio) mixture) to obtain 89.3 mg of the product (yield: 75.8%).

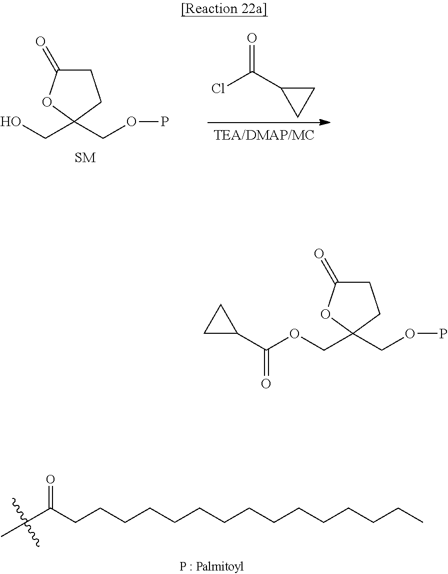

### [Example 23] Synthesis of Diacylglycerol Lactone Compound (EC-A135)

As shown in Reaction 23a below, 100 mg (0.26 mmole) of the product (SM) of Reaction 18b was dissolved in 1 ml of methylene chloride (MC). Then, 72.5 mL (0.52 mmole) of triethylamine (TEA) and 3.17 mg (0.026 mmole) of 4-dimethylaminopyridine (DMAP) were added and stirred. Then, it was stirred at 0° C. for 30 minutes. 45.2 mL (0.338 mmole) of cyclopropanecarbonyl chloride was added in the reaction solution and stirred at room temperature for 2 hours. The reaction was confirmed by TLC (EA:Hex=1:2). When the reaction was completed, it was extracted with 0.1N-hydrochloric acid solution (c-HCl soln.)/methylene chloride (MC) and then concentrated. The concentrate was purified with a flash column (eluent:ethyl acetate(EA):hexane(Hex)=1:4 (volume ratio) mixture) to obtain 88.1 mg of the product (yield: 68.5%).

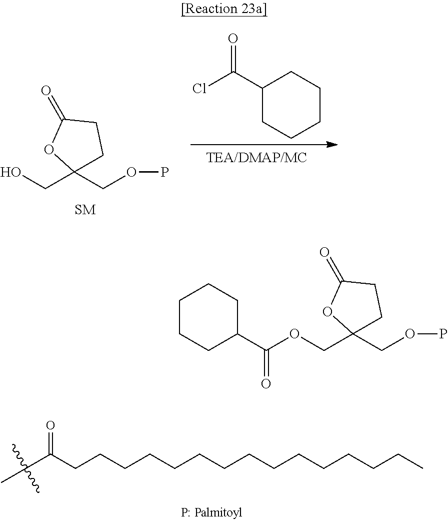

### [Example 24] Synthesis of Diacylglycerol Lactone Compound (EC-A136)

As shown in Reaction 24a below, 67.7 mg (0.338 mmole) of lauric acid and 42 mL (0.338 mmole) of pivaloyl chloride were added in 1 ml of methylene chloride (MC). Then, it was cooled at 0° C. 0.1 ml (0.78 mmole) of triethylamine (TEA) was slowly added dropwise at the same temperature. It was stirred at the same temperature for 30 minutes. 100 mg (0.26 mmole) of the product (SM) of Reaction 18b and 3.17 mg (0.026 mmole) of 4-dimethylaminopyridine (DMAP) were added at the same temperature. It was stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 103.6 mg of the target compound (yield: 70.3%).

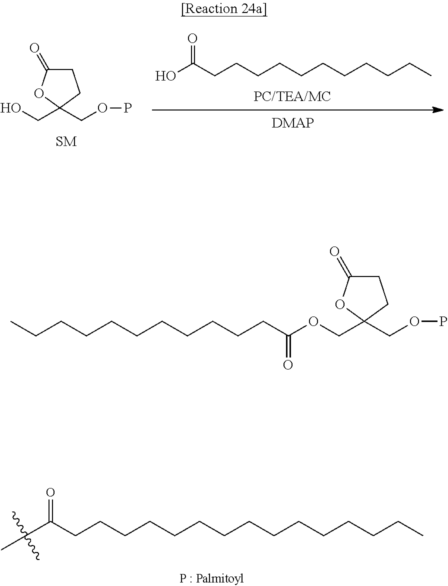

### [Example 25] Synthesis of Diacylglycerol Lactone Compound (EC-A137)

As shown in Reaction 25a below, 77.2 mg (0.338 mmole) of myristic acid and 42 mL (0.338 mmole) of pivaloyl chloride were added in 1 ml of methylene chloride (MC). Then, it was cooled at 0° C. 0.1 ml (0.78 mmole) of triethylamine (TEA) was slowly added dropwise at the same temperature. It was stirred at the same temperature for 30 minutes. 100 mg (0.26 mmole) of the product (SM) of Reaction 18b and 3.17 mg (0.026 mmole) of 4-dimethylaminopyridine (DMAP) were added at the same temperature. It was stirred at room temperature for overnight. The reaction was confirmed by TLC (EA:Hex=1:2). When the reaction was completed, it was extracted with 0.1N-potassium hydroxide solution (KOH soln.)/methylene chloride (MC). The extract solution was extracted with 0.07N-hydrochloric acid solution (c-HCl soln.)/methylene chloride (MC) and then concentrated after removing moisture with magnesium sulfate (MgSO₄). The concentrate was purified with a flash column (eluent:ethyl acetate(EA):hexane(Hex)=1:4 (volume ratio) mixture) to obtain 106.4 mg of the target compound (yield: 68.8%).

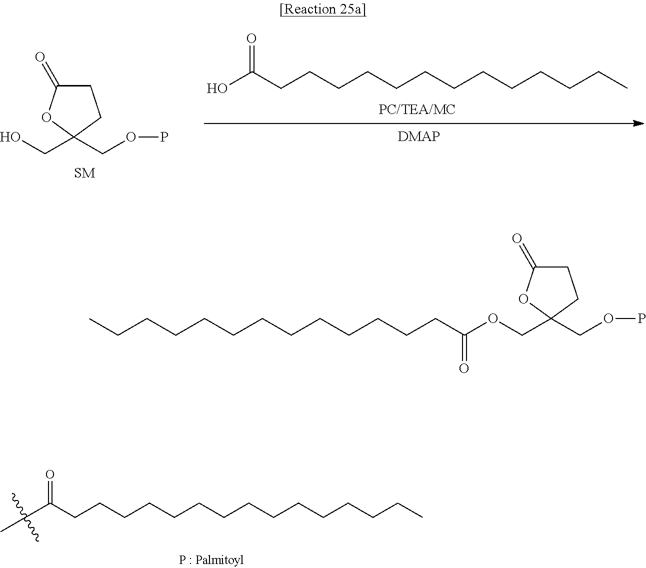

### [Experimental Example 1-1] Cytotoxicity Assessment of Diacylglycerol Lactone Compound

In DMEM (Dulbecco Modified Eagle Medium) medium with 10% Fetal Bovine Serum added, RAW264.7 cells, which are cells of the mouse macrophage family, were suspended at a concentration of 1×10⁵ cells/ml, and then were inoculated into 96 well plates by 100 μl, and culture was conducted for 15 hours. Next, the culture solution was treated with a diacylglycerol lactone compound having the kind and concentration as shown in Table 1 below, and then the additional culture was carried out for 24 hours. According to the EZ-CYTOX (Daeillab_EZ-1000) manual for measuring the amount of living cells using WST, 10 μl of EZ-Cytox was added to each well and reacted for up to 2 hours from 30 minutes, and then an optical density (OD) at 450 nm was measured. Cell viability was calculated according to Equation 1 below, and the results thereof are shown together in Table 1.

$$Cell viability ( \% ) = ( \frac{\begin{matrix}OD at 450 nm of Treatment group of \\ diacylglycerol lactone compound\end{matrix}}{OD at 450 nm of negative control group} ) \times 100 \qquad [ Equation 1 ]$$

TABLE 1

| | | | RAW267.4 |
|---|---|---|---|
| | | | cell viability |
| | | concentration | (%, average ± |
| | sample | (μg/ml) | deviation) |
| 1 | Negative control group | 0 | 107.09 ± 10.03 |
| 2 | EC-A115 | 100 | 145.03 ± 11.86 |
| 3 | EC-A116 | 100 | 141.09 ± 5.01 |
| 4 | EC-A117 | 100 | 111.93 ± 0.09 |
| 5 | EC-A118 | 100 | 126.45 ± 10.58 |
| 6 | EC-A119 | 100 | 116.45 ± 13.41 |
| 7 | EC-A120 | 100 | 106.83 ± 17.7 |
| 8 | EC-A121 | 100 | 114.25 ± 33.11 |
| 9 | EC-A122 | 100 | 105.48 ± 3.92 |
| 10 | EC-A123 | 100 | 107.35 ± 3.46 |
| 11 | EC-A124 | 100 | 146.77 ± 6.47 |
| 12 | EC-A125 | 100 | 106 ± 9.94 |
| 13 | EC-A126 | 100 | 107.74 ± 0.91 |
| 14 | EC-A127 | 100 | 110.25 ± 4.47 |
| 15 | EC-A128 | 100 | 129.35 ± 21.98 |
| 16 | EC-A129 | 100 | 103.93 ± 0.27 |
| 17 | EC-A130 | 100 | 118.51 ± 34.21 |
| 18 | EC-A131 | 100 | 100.25 ± 14.78 |
| 19 | EC-A132 | 100 | 96.58 ± 5.01 |
| 20 | EC-A133 | 100 | 114.45 ± 3.46 |
| 21 | EC-A134 | 100 | 110.06 ± 4.19 |
| 22 | EC-A135 | 100 | 104 ± 3.28 |
| 23 | EC-A136 | 100 | 95.54 ± 3.01 |
| 24 | EC-A137 | 100 | 93.87 ± 8.85 |

As shown in Table 1, from the results of observing the cell viability of RAW264.7 cells dependent on the diacylglycerol lactone compound of the present invention, it was confirmed that all compounds did not exhibit cytotoxicity at a concentration of 100 μg/ml.

### [Experimental Example 2-1] Increase in Expression of CXCL8 (IL-8) of Diacylglycerol Lactone Compound

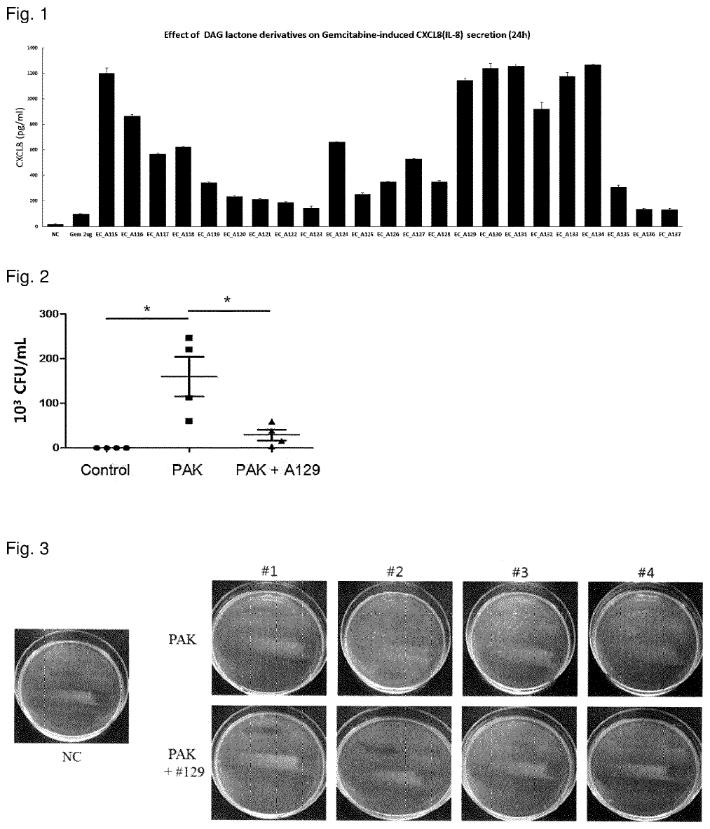

In RPMI (Hyclone, Thermo Scientific) medium to which 10% Fetal Bovine Serum was added, THP-1 cells, a human macrophage family, were suspended at a concentration of 1×10⁵ cells/ml, and culture was conducted in a 5% CO₂ humidified incubator at 37° C. The cultured THP-1 cells were inoculated into a 12 well plate by 1×106 cells/ml and stabilized for 30 minutes. Then, the culture solution was treated with a diacylglycerol lactone compound of the type shown in Table 2 below for 1 hour and then was treated with Gemcitabine (2 μg/ml) of a cell stimulator, and subsequent further incubation was conducted for 24 hours. Thereafter 1.5 ml of the culture supernatant was collected for each well and centrifuged (at 3000 rpm, 5 minutes) to recover the supernatant. The CXCL8 (IL-8) level in the recovered supernatant was measured according to the manual provided by the human IL-8 ELISA set (BD Biosciences). The day before ELISA was carried out, the IL-8 capture antibody was diluted in phosphate buffered saline, coated on a microwell, and then stored at 4° C. overnight. Each well was washed three times with a washing buffer solution and then blocked with 2% Bovine Serum Albumin (BSA) for 1 hour at room temperature. After washing with washing buffer solution three times, 100 μl of sample was dispensed into each well and left at room temperature for 2 hours. Detection antibody which was washed 3 times with washing buffer and diluted was dispensed into each well and allowed to react at room temperature for 1 hour and left at room temperature for 1 hour. Thereafter, the secondary HRP conjugated antibody was reacted at room temperature for 30 minutes, washed three times with a washing buffer, and treated with 50 μl of stop solution for each well, and then the optical density was measured at 450 nm with an ELISA microplate leader. The results of the expression increase rate are shown in Table 2 and FIG. 1 below.

TABLE 2

| | | | CXCL8 [IL-8] |
|---|---|---|---|
| | | | concentration |
| | | Concentration | (pg/μl, |
| | sample | (μg/ml) | average ± deviation) |
| 1 | Negative control group | 0 | 20.54 ± 2.50 |
| 2 | Gemcitabine | 2 | 101.83 ± 1.59 |
| 3 | EC-A115 | 100 | 1202.80 ± 38.09 |
| 4 | EC-A116 | 100 | 865.54 ± 12.31 |
| 5 | EC-A117 | 100 | 568.12 ± 8.21 |
| 6 | EC-A118 | 100 | 622.16 ± 6.61 |
| 7 | EC-A119 | 100 | 343.61 ± 7.75 |
| 8 | EC-A120 | 100 | 237.80 ± 3.64 |
| 9 | EC-A121 | 100 | 215.38 ± 4.33 |
| 10 | EC-A122 | 100 | 190.06 ± 4.56 |
| 11 | EC-A123 | 100 | 144.25 ± 17.33 |
| 12 | EC-A124 | 100 | 663.45 ± 2.96 |
| 13 | EC-A125 | 100 | 252 ± 11.86 |
| 14 | EC-A126 | 100 | 352.96 ± 0.45 |
| 15 | EC-A127 | 100 | 532.32 ± 1.82 |
| 16 | EC-A128 | 100 | 351.03 ± 7.75 |
| 17 | EC-A129 | 100 | 1145.87 ± 15.5 |
| 18 | EC-A130 | 100 | 1239.25 ± 39.91 |
| 19 | EC-A131 | 100 | 1256.19 ± 15.96 |
| 20 | EC-A132 | 100 | 921.35 ± 52.46 |
| 21 | EC-A133 | 100 | 1176.67 ± 29.42 |
| 22 | EC-A134 | 100 | 1266.03 ± 2.05 |
| 23 | EC-A135 | 100 | 310.87 ± 14.37 |
| 24 | EC-A136 | 100 | 138.93 ± 2.96 |
| 25 | EC-A137 | 100 | 133.12 ± 8.43 |

From Table 2 and FIG. 1, it was confirmed that when THP-1 cells are treated with Gemcitabine, an anticancer drug, the secretion of CXCL8 (IL-8) chemokine, a neutrophil cell recruitment factor, is increased by about five times compared to the negative control group, and addition of diacylglycerol lactone compound increase CXCL8 chemokine secretion from THP-1 cells by at least 1.3 times and up to 12 times with respect to that of the anticancer treatment group.

### [Experimental Example 3-1] Animal Model of Lung Infection with Bacteria and Sample Administration

For getting mice model whose lung are infected with bacteria, 12-week old Balb/c male mice were purchased from Koatech Corporation (South Korea) and maintained in certain pathogen-free facilities under moderate temperature and lights cycles. For obtain bacteria to induce lung infection, Aeruginosa K (PAK) of the genus Psuedomonas was incubated in LB broth or LB agar plate overnight at 37° C., and then the culture solution was centrifuged at 13,000×g for 2 minutes to obtain a bacterial pellet. Thereafter, the bacterial pellet was suspended in phosphate buffered saline (PBS), and the optical density of the serial dilution was measured and plated on an agar plate, so that bacterial inoculum having a colony forming unit (CFU) was obtained. For use in the following experiments, a bacterial inoculum solution for infection was prepared at a concentration of 1×10⁵ CFU per 20 μl.

### [Experimental Example 4-1] Confirmation of CFU Level in Mice Infected with P. aeruginosa

The PAK bacteria inoculum prepared in Experimental Example 3 (1×10⁵ CFU per mouse in 20 μl PBS) was administered to a total of eight 12-week-old Balb/c mice by nasal injection, wherein diacylglycerol lactone compound (EC_A129) was orally administered by 250 mg/kg to four mice among the PAK-treated groups, and PBS was administered to the control group. After 4 hours, samples of bronchoalveolar lavage fluid (BALF) were collected from the PAK-treated group. The collected BALF sample was diluted 1:1000 with PBS, and the diluted sample was plated on LB agar, and then incubated overnight at 37° C. CFU levels in BALF were confirmed by measuring the number of surviving bacteria by a plate count method, and the results thereof are shown in Table 3, FIGS. 2 and 3 below.

TABLE 3

| Mouse subject | Negative control | PAK infected group | PAK + diacylglycerol lactone compound |
|---|---|---|---|
| number | group (10³ CPU/ml) | (10³ CPU/ml) | EC-A129 treated group (10³ CPU/ml) |
| # 1 | 0 | 112.0 | 59.0 |
| # 2 | 0 | 246.0 | 3.0 |
| # 3 | 0 | 220.0 | 16.0 |
| # 4 | 0 | 60.0 | 38.0 |
| average ± deviation | 0 | 160.0 ± 88.0 | 29.0 ± 25.0 |

As shown in Table 3 and FIG. 2, it was confirmed that the bacterial CFU in the BALF was rapidly increased at 4 hours after PAK administration. On the other hand, when the A129 compound which significantly increased the CXCL8 expression, a neutrophil recruiting factor, among the diacylglycerol lactone compounds, was administered together with PAK, the bacterial CFU in the alveolar lavage fluid at 4 hours was significantly lower than in the PAK alone group. As described in Experimental Example 3, BALF collected for each mouse for each group was diluted 1:1000 with PBS and applied 100 μl to an LB plate to incubate the bacteria for 16 hours and the number of bacteria then was measured. Before measuring the number of bacteria, the LB plates were photographed, which is shown in FIG. 3, illustrating that in PAK-infected mice, diacylglycerol lactone compound promotes bacterial removal in the early stages of infection.

1. A diacylglycerol lactone compound represented by following Chemical formula 1, 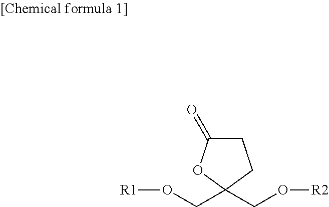
in Chemical formula 1, R1 and R2 are independently a fatty acid residue of 2 to 30 carbon atoms.

2. The diacylglycerol lactone compound of claim 1, wherein R1 and R2 are independently selected from the group consisting of acetyl, propionyl, butyryl, isobutyryl, valeroyl, pivaloyl, 2-methylbutyryl, cyclopropanecarbonyl, cyclohexanecarbonyl, hexanoyl, heptanoyl, nonanoyl, dodecanoyl, myristoyl, palmitoyl, linoleoyl, oleoyl, linolenoyl, eicosanoyl and arachidonoyl.

3. The diacylglycerol lactone compound of claim 1, wherein the diacylglycerol lactone compound of Chemical formula 1 is a compound represented by following Chemical formula 2a or Chemical formula 2b.

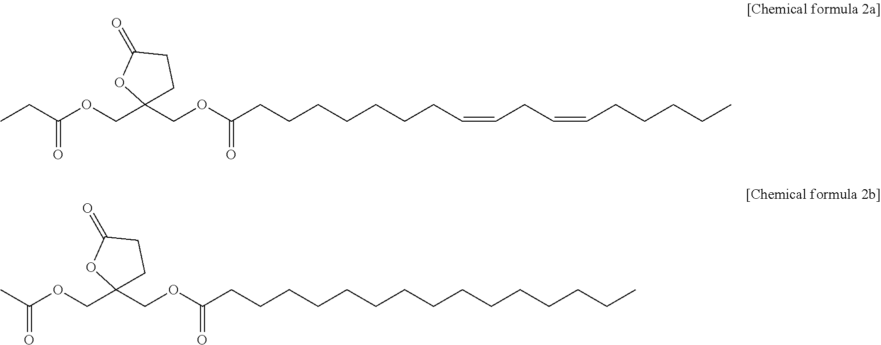

4. A method for preparing diacylglycerol lactone comprising the steps of:
reacting a compound represented by Chemical formula A with CH₂═CH—CH₂—MgX (X is a halogen atom) to prepare a compound represented by Chemical formula B, as shown in Reaction 1 below, 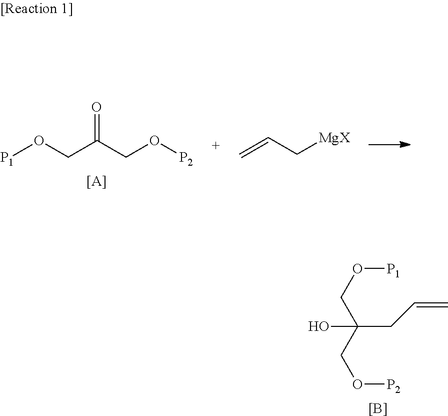
in Reaction 1, P1 and P2 are protecting groups;
carrying out hydroxylation of a double bond of the compound of Chemical formula B to obtain a compound represented by Chemical formula C, as shown in Reaction 2 below, 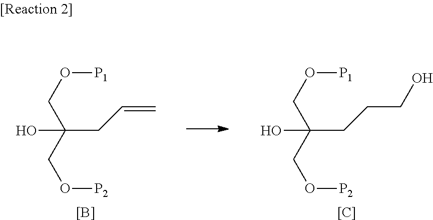
in Reaction 2, P1 and P2 are protecting groups;
carrying out lacton-cyclization reaction of the compound of Chemical formula C to obtain a compound represented by Chemical formula D, as shown in Reaction 3 below, 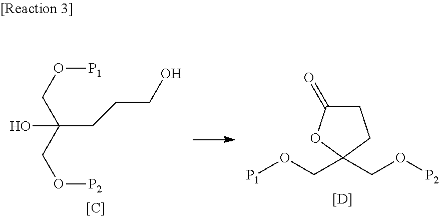
in Reaction 3, P1 and P2 are protecting groups; and
carrying out deprotection reaction and esterification reaction of the compound of Chemical formula D to obtain a compound represented by Chemical formula 1, as shown in Reaction 4 below, 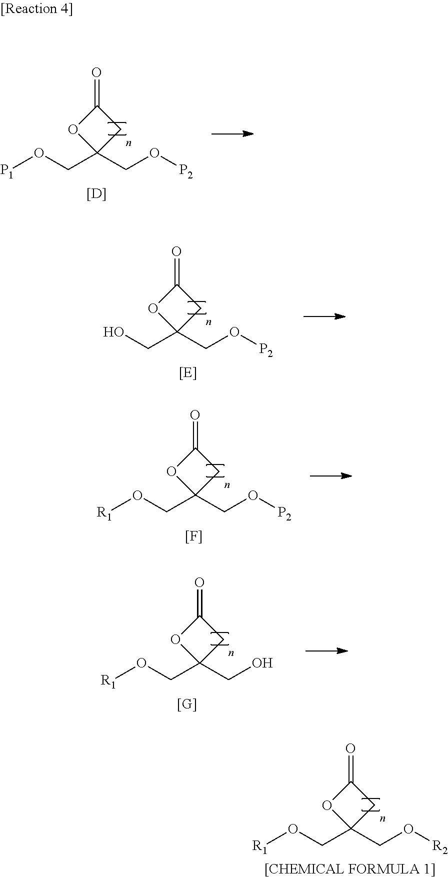
in Reaction 4, P1 and P2 are protecting groups, and R1 and R2 are independently a fatty acid residue of 2 to 30 carbon atoms.

5. An immunostimulator comprising diacylglycerol lactone compound represented by following Chemical formula 1 as an active ingredient, 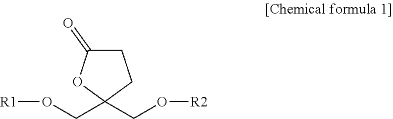
in Chemical formula 1, R1 and R2 are independently a fatty acid residue of 2 to 30 carbon atoms.

6. The immunostimulator of claim 5, wherein the diacylglycerol lactone compound increases IL-8 cytokine expression.

7. The immunostimulator of claim 5, wherein the diacylglycerol lactone compound promotes neutrophil migration from blood to cells.

8. The immunostimulator of claim 5, wherein the diacylglycerol lactone compound prevents or treats immune disease selected from the group consisting of bacterial or viral infections, acute and chronic inflammatory lung diseases, pneumonia and sepsis.

9. The immunostimulator of claim 5, wherein amount of the diacylglycerol lactone compound is 0.0001 to 100.0% by weight.

10. A health functional food composition for immunity enhancement, comprising diacylglycerol lactone compound represented by following Chemical formula 1 as an active ingredient, 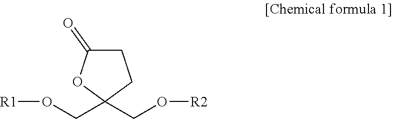
in Chemical formula 1, R1 and R2 are independently a fatty acid residue of 2 to 30 carbon atoms.

11. A method for immunity enhancement comprising administering to a non-human subject the immunostimulator according to claim 5.

12. A method for immunity enhancement comprising administering to a non-human subject the immunostimulator according to claim 6.

13. A method for immunity enhancement comprising administering to a non-human subject the immunostimulator according to claim 7.

14. A method for immunity enhancement comprising administering to a non-human subject the immunostimulator according to claim 8.

15. A method for immunity enhancement comprising administering to a non-human subject the immunostimulator according to claim 9.
